(19) United States
(12) Patent Application Publication
(10) Pub. No.: US 2011/0039903 A1
(43) Pub. Date: Feb. 17, 2011

(54) Acute Pharmacologic Augmentation of Psychotherapy with Enhancers of Learning or Conditioning

(76) Inventors: Michael Davis, Stone Mountain, GA (US); Kerry J. Ressler, Atlanta, GA (US)

Correspondence Address: JASON P. MCDEVITT, 124 COUNTRY CLUB DRIVE, WILLIAMSBURG, VA, 23188, US

(21) Appl. No.: 12/909,416

(22) Filed: Oct. 21, 2010

Related U.S. Application Data
(63) Continuation of application No. 10/924,591, filed on Aug. 24, 2004.
Application No. 10/924,591 is a continuation-in-part of application No. 10/473,640, filed on Apr. 22, 2004, now Pat. No. 7,750,030.
(60) Provisional application No. 60/363,991, filed on Mar. 13, 2002.
(60) Provisional application No. 60/279,868, filed on Mar. 29, 2001.

(51) Int. Cl. A61K 31/42; A61P 25/00

(52) U.S. Cl. 514/380

(57) ABSTRACT

Methods for treating an individual with a psychiatric disorder with a pharmacologic agent that enhances learning or conditioning in combination with a session of psychotherapy are provided. These methods of the invention encompass a variety of methods of psychotherapy, including exposure-based psychotherapy, cognitive psychotherapy, and psychodynamically oriented psychotherapy, and psychiatric disorders including fear and anxiety disorders, addictive disorders including substance-abuse disorders, and mood disorders. The pharmacologic agents used for the methods of the present invention are ones that generally enhance learning or conditioning, including those that increase the level of acetylcholine in the brain, and those that enhance N-methyl-D-aspartate (NMDA) receptor transmission in the brain.

### CROSS-REFERENCE TO RELATED APPLICATIONS

This application is a continuation of co-pending U.S. patent application Ser. No. 10/924,591, filed Aug. 24, 2004; which is a continuation-in-part of application Ser. No. 10/473,640, filed on Apr. 22, 2004, issued as U.S. Pat. No. 7,750,030; which is a national stage application under 35 U.S.C. 371 of International Application PCT/US02/09467, filed Mar. 28, 2002; which claims priority to U.S. Provisional Application No. 60/363,991, filed Mar. 13, 2002, and U.S. Provisional Application No. 60/279,868, filed Mar. 29, 2001; all of which are incorporated herein by reference in their entireties.

### STATEMENT REGARDING FEDERALLY SPONSORED RESEARCH OR DEVELOPMENT

This invention was made with U.S. Government support under grant MH057250 awarded by the National Institutes of Health. The U.S. Government may have certain rights in this invention.

### FIELD OF THE INVENTION

The invention relates to methods for treating an individual with a psychiatric disorder with a pharmacologic agent that enhances learning or conditioning in combination with psychotherapy.

### BACKGROUND OF THE INVENTION

Classical fear conditioning occurs when an affectively neutral stimulus is paired with a noxious aversive stimulus (unconditioned stimulus [US]) such as footshock. Afterward, the previously neutral stimulus (i.e., now the conditioned stimulus [CS]) is able to elicit a variety of autonomic, hormonal, and skeletal responses that accompany the conscious experience of fear in humans and which are used to operationally define fear in laboratory animals. The fear-eliciting properties of the CS can be extinguished by repeatedly presenting the CS in the absence of the US. It is generally believed that extinction does not reflect unlearning of the original association but involves instead the formation of new associations that compete with the previously conditioned response (see Bouton and Bolles (1985) Context, Event Memories, and Extinction (Lawrence Erlbaum Associates, Hillsdale, N.J.); Fall and Davis (1995) “Behavioral and Physiological Analysis of Fear Inhibition” in Neurobiological and Clinical Consequences of Stress: From Normal Adaptation to PTSD, eds. Friedman et al. (Lippincott-Raven Publishers, Philadelphia, Pa.); Davis et al. (2000) “Neural Systems Involved in Fear Lnhibition: Extinction and Conditioned Inhibition,” in Contemporary Issues in Modeling Psychopathology, eds. Myslobodsky and Weiner (Kluwer Academic Publishers, Boston, Mass.); Rescorla (2001) “Experimental Extinction,” in Handbook of Contemporary Learning Theories, eds. Mowrer and Klein (Erlbaum, Mahwah, N.J.)).

As with fear conditioning itself, fear extinction can be blocked by N-methyl-D-aspartate (NMDA) receptor antagonists administered either systemically (Cox and Westbrook (1994) Quarterly J. Exp. Psych. 47B:187-210; Baker and Azorlosa (1996) Behav. Neuroscience 110:618-620) or infused directly into the amygdala (Falls et al. (1992) J. Neuroscience 12:854-863; 1992; Lee and Kim (1998) J. Neuroscience 18:8444-8454). The involvement of the amygdala is of particular interest given the well known involvement of this structure in excitatory fear conditioning (Kapp et al. (1990) “A Neuroanatomical Systems Analysis of Conditioned Bradycardia in the Rabbit,” in Neurocomputation and Learning: Foundations of Adaptive Networks, eds. Gabriel and Moore (Bradford Books, New York); Fanselow and LeDoux (1999) Neuron 23:229-232; Davis (2000) “The Role of the Amygdala in Conditioned and Unconditioned Fear and Anxiety,” in The Amygdala, Volume 2, ed. Aggleton (Oxford University Press, Oxford, United Kingdom)).

Because NMDA receptor antagonists block extinction, it is possible that NMDA receptor agonists would facilitate extinction. However, the well-documented neurotoxic effects of NMDA receptor agonists argue against their use in humans. For example, increasing attention has focused on partial agonists that might facilitate NMDA receptor activity in a more limited fashion (Lawlor and Davis (1992) Biological Psychiatry 31:337-350; Olney (1994) J. Neural Transmission Suppl. 43:47-51). In fact, partial agonists such as D-Cycloserine (DCS), a compound that acts at the strychnine-insensitive glycine recognition site of the NMDA receptor complex, have been shown to enhance learning and memory in several animal paradigms including visual recognition tasks in primates (Matsuoka and Aigner (1996) J. Pharmacol. Exp. Ther. 278:891-897), eyeblink conditioning in rabbits (Thompson et al. (1992) Nature 359:638-641), avoidance learning in rats and mice (Monahan et al. (1989) Pharmacol., Biochem. Behav. 34:649-653; Flood et al. (1992) Eur. J. Pharmacol. 221:249-254; Land and Riccio (1999) Neurobiol. Learn. Mem. 72:158-168), and maze learning in rats and mice (Monahan et al. (1989) Pharmacol., Biochem. Behav. 34:649-653; Quartermain et al. (1994) Eur. J. Pharmacol. 257:7-12; Pitkanen et al. (1995) Eur. Neuropsychopharmacol. 5:457-463; Pussinen et al. (1997) Neurobiol. Learn. Mem. 67:69-74), without producing obvious neurotoxicity. DCS has also been found, in some studies, to modestly improve cognition in clinical populations (Javitt et al. (1994) Am. J. Psychiatry 151:1234-1236; Schwartz et al. (1996) Neurology 46:420-424; Goff et al. (1999) Arch. General Psychiatry 56:21-27; Tsai et al. (1999) Am. J Psychiatry 156:467-469), and has been used for many years to treat tuberculosis, again without obvious neurotoxicity.

A reduced ability to extinguish intense fear memories is a significant clinical problem for a wide range of psychiatric disorders including specific phobias, panic disorder, and post-traumatic stress disorder (see Morgan et al. (1995) Biol. Psychiatry 38:378-385; Fyer (1998) Biol. Psychiatry 44:1295-1304; Gorman et al. (2000) Am. J. Psychiatry 157:493-505). Because treatment for these disorders often relies upon the progressive extinction of fear memories (Zarate and Agras (1994) Psychiatry 57:133-141; Dadds et al. (1997) Psychological Bull. 122:89-103; Foa (2000) J. Clin. Psychiatry 61:43-48), pharmacological enhancement of extinction could be of considerable clinical benefit in these conditions.

### BRIEF SUMMARY OF THE INVENTION

Methods for treating a psychiatric disorder in an individual are provided. The methods comprise subjecting the individual in need of treatment to at least one session of a combination therapy protocol, where the protocol comprises administering a therapeutically effective amount of a pharmacologic agent that enhances learning or conditioning within about 24 hours prior to conducting a session of psychotherapy. Suitable pharmacologic agents that enhance learning or conditioning include pharmacologic agents that increase the level of norepinephrine in the brain, pharmacologic agents that increase the level of acetylcholine in the brain, and pharmacologic agents that enhance NMDA receptor transmission in the brain. The methods find use in the treatment of a variety of psychiatric disorders, including fear and anxiety disorders, addictive disorders, mood disorders, and movement disorders.

### BRIEF DESCRIPTION OF THE DRAWINGS

FIG. 1 shows the parametric evaluation of different amounts of extinction training. A. Timeline of the behavioral procedures for Experiment 1. B. Percent fear-potentiated startle measured 24 hrs before (pre-test) and 24 hrs after (post-test) extinction training or context exposure. The control group was tested 2 days after the pre-test, with no intervening exposures. One session of non-reinforced cue exposure produced only modest levels of extinction. Two or three sessions more completely extinguished the fear response. * p<0.05 versus context exposure group, +p<0.05 versus control group.

FIG. 2 shows the dose-response function for the effect of DCS on extinction. A. Timeline of the behavioral procedures for Experiment 2. B. Percent fear-potentiated startle measured 24 hrs before and 24 hrs after a single session of extinction training in rats injected with saline or DCS (3.25, 15, or 30 mg/kg, i.p.) 30 min prior to non-reinforced cue exposure. DCS dose-dependently facilitated extinction learning. * p<0.05 versus saline post-extinction.

FIG. 3 shows the effect of DCS in non-extinguished rats. A. Timeline of the behavioral procedures for Experiment 3. B. Percent fear-potentiated startle measured 24 hrs before and 24 hrs after extinction training. Saline or DCS (15 mg/kg, i.p.) was administered 30 min prior to a single session of either extinction training (cue exposure) or context alone exposure. Fear-potentiated startle was significantly lower in rats that received DCS+extinction training than in rats that received saline+extinction training. Fear-potentiated startle was not appreciably affected by DCS in rats that did not receive extinction training. * p<0.05 versus saline+extinction training.

FIG. 4 shows the effect of the strychnine-insensitive glycine recognition site antagonist HA-966 on extinction and on the facilitation of extinction by DCS. A. Timeline of the behavioral procedures for Experiment 4. B. Percent fear-potentiated startle measured 24 hrs before (pre-extinction test) and 24 hrs after (post-extinction test) extinction training. Saline or HA-966 (6 mg/kg, i.p.) were administered 10 min before a second injection of saline or DCS, followed 30 min later by a single session of extinction training. HA-966 completely blocked the effects of DCS but did not, on its own, noticeably influence extinction at this dose. * p<0.05 versus all other groups.

FIG. 5 shows the effect of pre-test DCS and HA-966 administration on fear-potentiated startle. A. Timeline of the behavioral procedures for Experiment 5. B. Percent fear-potentiated startle measured 24 hrs after fear-conditioning in rats receiving pre-test injections of saline, DCS (15 mg/kg), or HA-966 (6 mg/kg). Neither drug had any discernible effect on fear-potentiated startle.

FIG. 6 shows cannula tip placements transcribed onto atlas plates adapted from Paxinos and Watson ((1997) The Rat Brain in Stereotaxic Coordinates (3^{rd} ed., Academic Press, New York)). The distance from bregma is indicated to the left; nuclei within the plane of section are identified to the right. BM=basomedial amygdaloid nucleus; BL=basolateral amygdaloid nucleus; BLV=basolateral amygdaloid nucleus, ventral part; CeM=central amygdaloid nucleus, medial division; CeL=central amygdaloid nucleus, lateral division; ic=internal capsule; LA=lateral amygdaloid nucleus; OPT=optic tract.

FIG. 7 shows the effect of intra-amygdala DCS infusions. A. Timeline of the behavioral procedures for Experiment 3. B. PBS or D-Cycloserine (10 μg/side) was infused into the amygdala 15 min prior to extinction training. Other rats received DCS without extinction training. When tested 24 hrs later, fear-potentiated startle was significantly lower in rats that received DCS+extinction training than in rats that received PBS+extinction training. Fear-potentiated startle was not appreciably affected by DCS in rats that did not receive extinction training. For the group that received DCS without extinction training, mean percent potentiation was calculated with and without data from a single outlier who had an atypically high percent potentiation score. * p<0.05 versus all other groups.

FIG. 8 is a composite figure showing absolute startle values for all rats receiving drugs prior to extinction training. Dark bars indicate baseline startle amplitude on noise alone trials; open bars indicate startle amplitude on light-noise trials. The difference between these two (i.e., fear-potentiated startle) is indicated by the striped bars. In no case were significant differences found in baseline startle during the fear-potentiated startle test 24 hrs after drug administration. Moreover the statistical results were similar when absolute difference scores (i.e., startle amplitude on light-noise trials minus startle amplitude on noise alone trials) rather than percent potentiation scores were analyzed. * p<0.05 (except Panel C, p=0.087) versus all left-most bars.

FIG. 9 shows measures of acrophobia within the virtual environment. A. Level of fear as measured by subjective units of discomfort (SUDS 1=no fear, 100=maximum fear) during the pre-treatment assessment at each successive floor in the virtual glass elevator. B. SUDS during the first treatment session in which subjects elevated to successive floors at 5-minute intervals. C. Floor to which the subjects elevated at 5-minute intervals during the first treatment session. There were no significant differences between the groups during the pretreatment SUDS measure or either measure during the first treatment session.

FIG. 10 shows improvement of acrophobia within the virtual environment with D-Cycloserine. A. Reduction in fear from pre to post-test following the two therapy sessions measured at the first follow-up assessment. Decrease in SUDS level (y-axis) is shown for each floor (1-19) of the virtual glass elevator. Overall ANOVA was performed using pre-post difference and floor as within-subjects variables and drug group as between-subjects variable. Significant overall pre-post changes were seen: F(1,25)=38, p≦0.001. Significant effect of floor was found: F(6, 150)=89, p≦0.001. Most importantly, significant effect of pre-post X floor X drug interaction was found: F(6,150)=3.8, p≦0.001. B. Change in SUDS from pre to post-test at the 3-month long-term follow-up assessment. Statistics were performed as above. Significant overall pre-post changes were seen: F(1,17)=21, p≦0.001. Significant effect of floor was found: F(6, 102)=81, p≦0.001. Most importantly, significant effect of pre-post X floor X drug interaction was found: F(6,102)=2.4, p≦0.05.

FIG. 11 shows physiological measures of anxiety within the virtual environment. Spontaneous fluctuations in baseline skin conductance levels are shown as a function of acrophobia treatment response and treatment condition. A. Subjective improvement in acrophobia symptoms. Those reporting improvement in symptoms show significantly lower post-treatment spontaneous fluctuations in the virtual environment (F(1,19)=4.5, p≦0.05). B. Decreased avoidance (self-reports of whether they have self-exposed to heights since treatment) also was associated with significantly lower spontaneous fluctuations of skin conductance (F(1,19)=8.26; p≦0.01). C. Subjects treated with DCS during exposure therapy showed significant decreases in post-treatment fluctuations (paired t-test, p≦0.05) compared to those treated with placebo (p≧0.5).

FIG. 12 shows reduction in fear compared to pretreatment baseline on general measures of acrophobia in the real world, 1 week after the first therapy session (mid-treatment), 1-2 weeks after the second therapy session (Post 1 week), or at 3-month follow-up (Post 3 month). A. Acrophobia Avoidance Questionnaire, (repeated measures ANOVA, DCS vs Placebo: F(1,19)=6.1, p≦0.02). B. Acrophobia Anxiety Questionnaire, (repeated measures ANOVA, DCS vs Placebo: F(1,19)=7.9, p≦0.01). C. Attitude Towards Heights Inventory, (repeated measures ANOVA, DCS vs Placebo: F(1,19)=4.9, p≦0.04).

FIG. 13 shows measures of global improvement and self exposure. A. Average Clinical Global Improvement scores (CGI, 1=“Very much improved”, 4=“No Change”) for placebo vs. DCS groups at 1 week and 3 months following treatment. (repeated measures ANOVA, DCS vs Placebo: F(1,19)=11.6, P<0.01). B. Percentage of subjects rating themselves as “Very Much Improved” or “Much Improved” on the CGI. Subjects receiving DCS during treatment demonstrated significantly greater subjective improvement compared to those receiving placebo. (repeated measures ANOVA, DCS vs placebo demonstrating an overall drug effect but no drug x time interaction; F(1,19)=11.5, p≦0.01). C. Reduction in acrophobia as measured by real-world self-exposures to heights during the 3 months following treatment. Subjects receiving DCS during treatment demonstrated significantly more exposures to heights at 3 months than did subjects receiving placebo (F(1,18)=7.7, p≦0.01).

### DETAILED DESCRIPTION OF THE INVENTION

The present invention is directed to methods for treating an individual with a psychiatric disorder. The methods comprise subjecting the individual to one or more sessions of a combination therapy protocol, where the combination therapy protocol comprises an acute administration of a therapeutically effective amount of a pharmacologic agent that enhances learning or conditioning in combination with a session of psychotherapy. By “acute administration” is intended a single exposure of the individual to the therapeutically effective amount of the pharmacologic agent that enhances learning or conditioning, where exposure to the pharmacologic agent occurs within about 24 hours prior to initiating the session of psychotherapy, preferably within about 12 hours, and more preferably within about 6 hours prior to initiating the session of psychotherapy. A full course of treatment for the psychiatric disorder entails at least one session of this combination therapy protocol.

As used herein, “psychiatric disorder” refers to a disorder that can be treated with the methods of the invention. For purposes of the present invention, an individual said to have a psychiatric disorder will have one or more disorders that can be treated with the methods of the invention. Thus an individual may have a single disorder, or may have a constellation of disorders that are to be treated by the methods described herein.

The psychiatric disorders contemplated in the present invention include, but are not limited to, fear and anxiety disorders, addictive disorders including substance-abuse disorders, and mood disorders. Within the fear and anxiety disorder category, the invention encompasses the treatment of panic disorder, specific phobia, post-traumatic stress disorder (PTSD), obsessive-compulsive disorder, and movement disorders such as Tourette's syndrome. The disorders contemplated herein are defined in, for example, the DSM-IV (Diagnostic and Statistical Manual of Mental Disorders (4th ed., American Psychiatric Association, Washington D.C., 1994)), which is herein incorporated by reference.

Any pharmacologic agent that is recognized by the skilled artisan as being a pharmacologic agent that enhances learning or conditioning can be used in the methods of the invention. For example, one such class of pharmacologic agents contemplated herein comprises compounds that increase the level of norepinephrine in the brain. Such compounds include those acting as norepinephrine reuptake inhibitors, for example tomoxetine, reboxetine (Edronax or Vestra), duloxetine, venlafaxine (Effexor®), and milnacipran (see, for example, U.S. Pat. No. 6,028,070, the contents of which are herein incorporated by reference), and those compounds that cause release of norepinephrine, for example amphetamine, dextroamphetamine (Dexedrine®), pemoline (Cylert®), and methylphenidate (Ritalin®). Another class of such pharmacologic agents are those compounds that increase the level of acetylcholine in the brain, including, for example, compounds that block its breakdown. Examples of such compounds include, but are not limited to, donepezil HCl or E2020 (Aricept®) and tacrine (THA, Cognex®), which inhibit cholinesterase activity.

Of particular interest are those pharmacologic agents that enhance N-methyl-D-aspartate (NMDA) receptor activation or transmission (cation flow) in the brain without adverse consequences such as neurotoxic effects. Such enhanced NMDA receptor transmission can be measured by a variety of methods known to the skilled artisan. In one embodiment, for example, Luteinizing Hormone (LH) secretion is used as a measure of NMDA receptor activation (see van Berckel et al. (1997) Neuropsychopharm. 16(5):317-324). Other methods include electrophysiological and chemical methods (see Mothet et al. (2000) Proc. Natl. Acad. Sci. USA 97(9):4926-4931). Neurotoxicity can be measured by, for example, the cultured cerebellar granule neuron system described in Boje et al. (1993) Brain Res. 603(2):207-214.

As used herein, the term “NMDA receptor” or “NMDA channel” refers to the glutamate receptor channel NMDA subtype (Yamakura and Shimoji (1999) Prog. Neurobiol. 59(3):279-298). The term “agonist” encompasses any compound that increases the flow of cations through an ionotrophic receptor such as the NMDA receptor, i.e., a channel opener, and which has not been observed to decrease the flow of cations through the same receptor. “Antagonist” includes any compound that reduces the flow of cations through an ionotropic receptor such as the NMDA receptor, i.e., a channel closer, and which has not been observed to increase the flow of cations through the same receptor. The term “partial agonist” refers to a compound that regulates an allosteric site on an ionotropic receptor, such as the NMDA receptor, to increase or decrease the flux of cations through the ligand-gated channel depending on the presence or absence of the principal site ligand, that is, in the presence or absence of a known endogenous ligand binding to a site on the receptor. In the absence of the principal site ligand, a partial agonist increases the flow of cations through the ligand-gated channel, but at a lower flux than achieved by the principal site ligand. A partial agonist partially opens the receptor channel. In the presence of the principal site ligand, a partial agonist decreases the flow of cations through the ligand-gated channel below the flux normally achieved by the principal site ligand.

As used herein, “NMDA receptor agonist,” “NMDA receptor antagonist,” and “NMDA receptor partial agonist,” may be alternately referred to as “NMDA agonist,” “NMDA antagonist,” and “NMDA partial antagonist,” respectively. Also, “NMDA receptor partial agonist” is intended to be interchangeable with “partial NMDA receptor agonist.” The present invention contemplates a variety of molecules acting as such partial NMDA receptor agonists. Examples of such pharmacologic agents include, but are not limited to, compounds that act at the glycine modulatory site of the NMDA receptor (see Yamakura and Shimoji (1999) Prog. Neurobiol. 59(3):279-298), including D-cycloserine (DCS)(see U.S. Pat. Nos. 5,061,721 and 5,260,324), D-serine, and 1-aminocyclopropane-carboxylic acid (ACPC)(see U.S. Pat. Nos. 5,086,072 and 5,428,069, herein incorporated by reference). Other pharmacologic agents that act as partial NMDA agonists, including polyamines such as spermine and spermidine, are also suitable for use in the methods of the present invention (Yamakura and Shimoji (1999) Prog. Neurobiol. 59(3):279-298).

The methods of the invention encompass the use of any type of psychotherapy that is suitable for the particular psychiatric disorder for which the individual is undergoing treatment. Suitable methods of psychotherapy include exposure-based psychotherapy, cognitive psychotherapy, and psychodynamically oriented psychotherapy. See, for example, Foa (2000) J. Clin. Psych. 61(suppl. 5):43-38.

One method of psychotherapy specifically contemplated is the use of virtual reality (VR) exposure therapy to treat a psychiatric disorder using the combination therapy protocol of the invention. VR exposure therapy has been used to treat a variety of disorders including anxiety disorders such as the fear of heights (Rothbaum and Hodges (1999) Behav. Modif. 23(4):507-25), as well as specific phobias, eating disorders, and PTSD (Anderson et al. (2001) Bull. Menninger Clin. 65(1):78-91). Because of the prevalence of PTSD in the general population and the successful use of VR therapy to treat PTSD in, for example, Vietnam veterans (Rothbaum et al. (1999) J. Trauma Stress 12(2):263-71) or rape victims (Rothbaum et al. (2001) J. Trauma Stress 14(2):283-93), one embodiment of the present invention specifically contemplates the use of such VR exposure psychotherapy in combination with a pharmacologic agent as described elsewhere herein to treat PTSD.

The timing of administration and therapeutically effective amount or dose of the particular pharmacologic agent used will depend on the pharmacologic agent itself, with the particular timing and dose selected in order to ensure that a therapeutically effect level of the pharmacologic agent is present in the individual being treated at the time of psychotherapy. In general, the timing of administration will be within about 24 hours before psychotherapy, more preferably within about 12 hours, and still more preferably within about 6 hours. A “therapeutically effective amount” or “therapeutically effective dose” of the pharmacologic agent is that amount of the pharmacologic agent that, when administered in accordance to the combination therapy protocol of the invention, results in an improved therapeutic benefit relative to that observed with psychotherapy in the absence of administering the pharmacologic agent. For example, where the pharmacologic agent is an agent that enhances NMDA receptor activation or transmission in the brain, a therapeutically effective dose or amount is that amount of the pharmacologic agent that enhances NMDA receptor activation or transmission in the brain relative to the level of NMDA receptor activation or transmission in the brain in the absence of administration of the pharmacologic agent. Similarly, when the pharmacologic agent is an agent that increases the level of norepinephrine or acetylcholine in the brain, a therapeutically effective dose or amount is that amount of the pharmacologic agent that increases the level of norepinephrine or acetylcholine in the brain relative to the level of these respective compounds in the brain in the absence of the administration of the pharmacologic agent.

For D-cycloserine, a preferred time of administration is within about 3-8 hours before psychotherapy. For this pharmacologic agent, dosage levels include a low dose level of between about 30-100 mg, and a high dose level of between about 400-500 mg. In one embodiment, D-cycloserine is administered in combination with D-alanine to minimize any potential gastrointestinal effects of this pharmacologic agent. See U.S. Pat. Nos. 5,061,721 and 5,260,324, herein incorporated by reference.

The therapeutically effective dose of the pharmacologic agent can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

A subject undergoing treatment with the methods of the invention exhibits an improvement in one or more symptoms associated with the psychiatric disorder. For a description of the relevant symptoms, see, for example, the DSM-IV ((1994) Diagnostic and Statistical Manual of Mental Disorders (4th ed., American Psychiatric Association, Washington D.C.)), which is herein incorporated by reference. The efficacy of the methods of the invention can be assessed using any clinically recognized assessment method for measuring a reduction of one or more symptoms of the particular psychiatric disorder. Examples of such assessment methods are described in, for example, Experiment 7, provided below.

The present invention may be better understood with reference to the following examples. These examples are intended to be representative of specific embodiments of the invention, and are not intended as limiting the scope of the invention.

### EXPERIMENTAL

Experiments 1-6 were conducted to examine the effects of the partial NMDA receptor agonist D-cycloserine on conditioned fear extinction. These experiments were conducted using Adult male Sprague-Dawley rats as described in the Materials and Methods section below. Experiments 7 and 8 describe a clinical trial of D-cycloserine augmentation of behavioral exposure therapy for human subjects suffering from a specific phobia.

### Materials and Methods for Experiments 1-6

### Animals

Adult male Sprague-Dawley rats (Charles River, Raleigh, N.C.) weighing between 300 and 400 g were used. Animals were housed in group cages of four rats each in a temperature (24° C.) controlled animal colony, with continuous access to food and water. They were maintained on a 12:12 light-dark cycle with lights on at 0700 hrs. All behavioral procedures took place during the rats' light cycle. A total of 178 rats were used.

### Apparatus

Animals were trained and tested in 8×15×15-cm Plexiglas and wire mesh cages. The cage floor consisted of four 6.0-mm diameter stainless steel bars spaced 18 mm apart. Each cage was suspended between compression springs within a steel frame and located within a custom-designed 90×70×70-cm ventilated sound-attenuating chamber. Background noise (60 dB wide-band) was provided by a General Radio Type 1390-B noise generator (Concord, Mass.) and delivered through high frequency speakers (Radio Shack Supertweeter; Tandy, Fort Worth, Tex.) located 5 cm from the front of each cage. Sound level measurements (SPL) were made with a Bruel & Kjaer (Marlborough, Mass.) model 2235 sound-level meter (A scale; random input) with the microphone (Type 4176) located 7 cm from the center of the speaker (approximating the distance of the rat's ear from the speaker).

Startle responses were evoked by 50-ms 95-dB white-noise bursts (5 ms rise-decay) generated by a Macintosh G3 computer soundfile (0-22 kHz), amplified by a Radio Shack amplifier (100 Watt; Model MPA-200; Tandy, Fort Worth, Tex.), and delivered through the same speakers used to provide background noise. An accelerometer (model U321A02; PCB Piezotronics, Depew, N.Y.) affixed to the bottom of each cage produced a voltage output proportional to the velocity of cage movement. This output was amplified (PCB Piezotronics, Model 483B21) and digitized on a scale of 0-2500 units by an InstruNET device (GW Instruments, Model 100B; Somerville, Mass.) interfaced to a Macintosh G3 computer. Startle amplitude was defined as the maximal peak-to-peak voltage that occurred during the first 200 ms after onset of the startle-eliciting stimulus.

The CS was a 3.7-s light (82 lux) produced by an 8-W fluorescent bulb (100-μs rise time) located 10 cm behind each cage. Luminosity was measured using a VWR light meter (Atlanta, Ga.). The unconditioned stimulus was a 0.5-s shock, delivered to the floorbars, and produced by a LeHigh Valley shock generator (SGS-004; LeHigh Valley, Beltsville, Md.). Shock intensities (measured as in Cassella et al. (1986) Physiol. Behav. 36:1187-1191) were 0.4 mA. The presentation and sequencing of all stimuli were under the control of the Macintosh G3 computer using custom-designed software (The Experimenter, Glassbeads Inc.; Newton, Conn.).

### Surgery and Histology

Rats that were to receive intra-amygdala infusions (Experiment 6) were anesthetized with Nembutal (sodium pentobarbital, 50 mg/kg, i.p) and placed in a stereotaxic frame (ASI Instruments, Inc., Warren, Mich.). The skull was exposed and 22-gauge guide cannulae (model C313G, Plastics One, Inc.; Roanoke, Va.) were implanted bilaterally into the basolateral nucleus of the amygdala (AP=−2.8; DV=−9.0; ML=+5.0 from bregma). Dummy Cannulae (model C313DC, Plastics One, Inc.) were inserted into each cannula to prevent clogging. These extended approximately 1 mm past the end of the guide cannula. Screws were anchored to the skull and the assembly was cemented in place using dental cement (The Hygenic Corp., Akron, Ohio).

Behavioral procedures began either 10 or 11 days after surgery. Cannulated rats subsequently received a chloral hydrate overdose and were perfused intracardially with 0.9% saline followed by 10% formalin. The brains were removed and immersed in a 30% sucrose-formalin solution for at least 3 d, after which 40-μm coronal sections were cut through the area of interest. Every fourth section was mounted and stained with cresyl violet.

### Drug Administration

Systemic administration: D-Cycloserine (Sigma-Aldrich, St. Louis, Mo.)—(3.25, 15, and 30 mg/kg)—and (±)-HA-966 (Research Biochemicals, Inc., Natick, Mass.)—(6 mg/kg) were freshly dissolved in saline and injected intraperitoneally 30 min prior to extinction training. Drug doses were chosen based on preliminary findings (data not shown), on the results of other behavioral studies (e.g., Monahan et al. (1989) Pharmacol. Biochem. Behav. 34:649-653; Flood et al. (1992) Eur. J. Pharmacol. 221:249-254; Moraes Ferreira and Morato (1997) Alcohol Clin. Exp. Res. 21:1638-1642; Pussinen et al. (1997) Neurobiol. Learn. and Mem. 67:69-74; Land and Riccio (1999) Neurobiol. Learn. Mem. 72:158-168), on estimates of brain concentration following systemic administration (extrapolated from Loscher et al. (1994) Brit. J. Pharmacol. 112:97-106) together with findings relating drug concentrations in vitro to DCS effects on NMDA receptor function measured electrophysiologically (e.g., Watson et al. (1990) Brain Res. 510:158-160; Priestley and Kemp (1994) Molecular Pharmacol. 46:1191-1196) or vis-à-vis ligand binding to the use-dependent channel-associated binding site (Hood et al. (1989) Neuroscience Letters 98:91-95; Hamelin and Lehmann (1995) Eur. J. Pharmacol. 281:R11-13), and on the ability of systemically administered DCS to influence NMDA receptor-mediated cGMP concentrations in mouse cerebellum (Emmett et al. (1991) NeuropharmacoL 30:1167-1171).

Intra-Amygdala Infusion: DCS (10 mg/side) or saline was infused (0.25 μl/min) through 28-gauge injection cannulas (model C313I, Plastic Products) 20 min prior to extinction training. The total volume infused was 0.5 μl/side. The infusion cannulae were left in place for 2 minutes before being withdrawn.

### General Behavioral Procedures

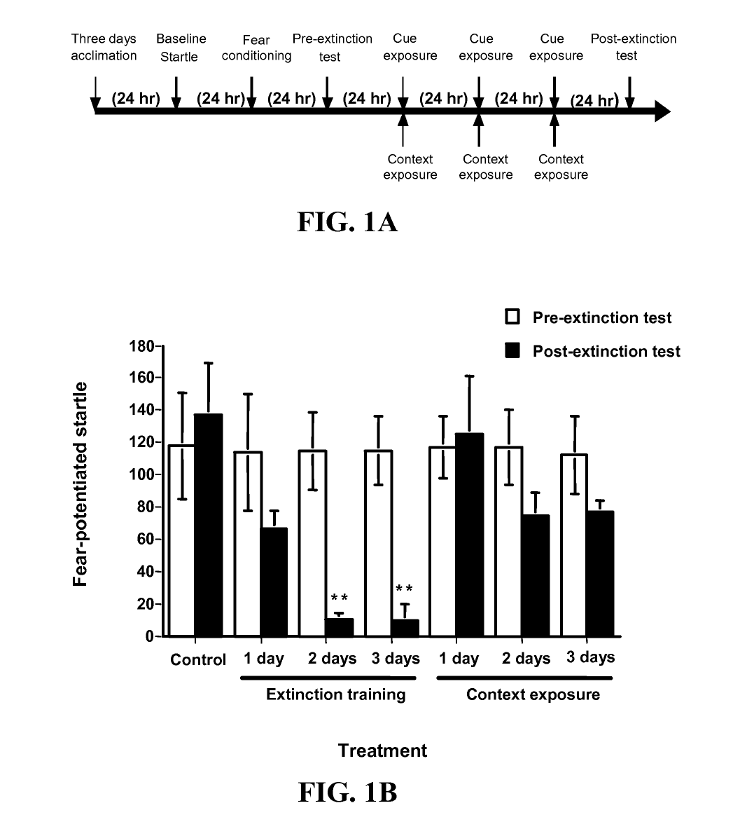

Behavioral procedures for all experiments consisted of an acclimation phase, a baseline startle test, a fear conditioning phase, a pre-extinction test, extinction training, and a post-extinction test (see FIG. 1A).

Acclimation. On each of three consecutive days, rats were placed into the test chambers for 10 min and then returned to their home cages.

Baseline startle test. On each of the next two consecutive days, animals were placed in the test chambers and presented with 30 95-dB noise bursts at a 30-s interstimulus interval (ISI). Animals whose baseline startle was less than 1% of the possible accelerometer output were excluded insofar as fear-potentiated startle cannot be properly measured with such a low baseline (a total of 2 rats out of 144 were excluded on this basis).

Fear conditioning. 24 hrs later, rats were returned to the test chambers and 5 minutes later given the first of 10 light-footshock pairings. The 0.4-mA 0.5-s shock was delivered during the last 0.5 sec of the 3.7-sec light. The average intertrial interval was 4 min (range=3-5 min).

Pre-extinction test. 24 hrs after fear conditioning, rats were returned to the test chambers and 5 min later were presented with 30 95-dB noise bursts (30-s ISI). These initial startle stimuli were used to habituate the startle response to a stable baseline prior to the noise alone and light-noise test trials that followed. A stable baseline, in turn, reduces variability in the fear-potentiated startle measure described below. Thirty seconds later, 20 additional noise bursts were presented (ISI=30 s). Half of these were presented in darkness (noise alone test trial) and half were presented 3.2 s after onset of the 3.7-s light (light-noise test trial). The order of these two trial types was randomized with the constraint that no two trial types occurred more than twice in a row. Percent fear-potentiated startle was computed as [(startle amplitude on light-noise minus noise-alone trials)/noise-alone trials]×100. Based on these data, rats were sorted into equal size groups such that each group had comparable mean levels of percent fear-potentiated startle. Because the fear-potentiated startle test is itself an extinction procedure (i.e., CS presentations without shock), and because we wanted to minimize any incidental extinction prior to explicit extinction training with drug, a minimal number of CS presentations was used in this test compared to the more lengthy post-extinction test described below. We have found, however, that this abbreviated test is adequate for matching rats into different groups with comparable levels of fear-potentiated startle.

Extinction training. 24 hrs after the pre-extinction test, rats were returned to the test chamber and 5 min later received 30 3.7-s light exposures without shock (ISI=30 s). Control rats were placed in the test cages and remained there for the same amount of time as rats in the extinction groups, but did not receive non-reinforced CS presentations. Rats in Experiment 1 received either 1, 2, or 3 sessions of extinction training with a 24-hr interval between each. Rats in all other experiments received a single session of extinction training.

Post-extinction test. 24 hrs after the last extinction session, rats were returned to the test chamber and, 5 min later, were presented with 30 95-dB noise bursts, as in the pre-extinction short-test, to habituate the startle response to a stable baseline prior to the noise alone and light-noise test trials that followed. 30 s later, 60 inter-mixed noise alone and light-noise test trials (95 dB, ISI=30 s) were presented. Percent potentiated startle was calculated from the noise alone and light-noise test trials as previously described.

### Statistics

ANOVA on percent fear-potentiated startle scores was the primary statistical measure. Between group comparisons were also made using two-tailed t-tests for independent samples. The criterion for significance for all comparisons was p <0.05.

### Results

### Experiments 1-6

### Experiment 1

### Parametric Evaluation of Different Amounts of Extinction Training

This experiment assessed the effect on fear-potentiated startle of 1, 2, or 3 days of extinction training. 42 rats were matched into 7 groups of 6 animals each based on their level of fear-potentiated startle in the pre-extinction test. Beginning 24 hrs after the pre-extinction test, rats received 1, 2, or 3 consecutive days of extinction training (30 non-reinforced light presentations per day), or 1, 2, or 3 days of exposure to the context without extinction training. An additional control group was tested 2 days after the pre-extinction test without intervening exposures to either context or the visual CS.

FIG. 1B shows that after 1 day of extinction training, fear-potentiated startle was reduced by approximately 35% compared to the pre-extinction test. After 2 or 3 days, fear-potentiated startle was reduced by approximately 90%. A two-way ANOVA with Treatment (non-reinforced CS presentations versus context exposure alone) and Days (one, two, or three extinction sessions) as between-subjects factors indicated a significant Treatment effect, F(1, 30)=13.01, and also a significant Treatment X Days interaction, F(2, 30)=8.90. Thus, the reduction of fear-potentiated startle across days was greater in the groups that received non-reinforced CS exposures than in the groups that received context exposure alone. Individual comparisons between non-reinforced CS presentation and context-exposure groups indicated significant differences after 2, t(10)=3.41, and after 3, t(10)=6.37, days. Significant differences versus the non-exposed control group were found versus rats that received one, t(10)=2.30, two, t(10)=4.33, or three, t(10)=4.26, days of extinction training.

### Experiment 2

### Dose-Response Function for the Effect of DCS on Extinction

Twenty-seven rats were acclimated, tested for baseline startle, fear-conditioned, and tested for fear-potentiated startle as previously described. Rats were then divided into 4 groups of 7 animals each (except the DCS 30 mg/kg where N=6] based on their pre-extinction level of fear-potentiated startle. 24 hrs later, each rat was injected with either saline or DCS (3.25, 15, or 30 mg/kg; i.p.). Thirty min later, rats received a single session of extinction training. A single extinction session was used because the results of Experiment 1 indicated that this produced a minimal amount of extinction against which a facilitatory effect of DCS could be detected. Twenty-four hours later, rats were tested for fear-potentiated startle without drug injections in order to evaluate the effect on extinction of the previous drug treatments.

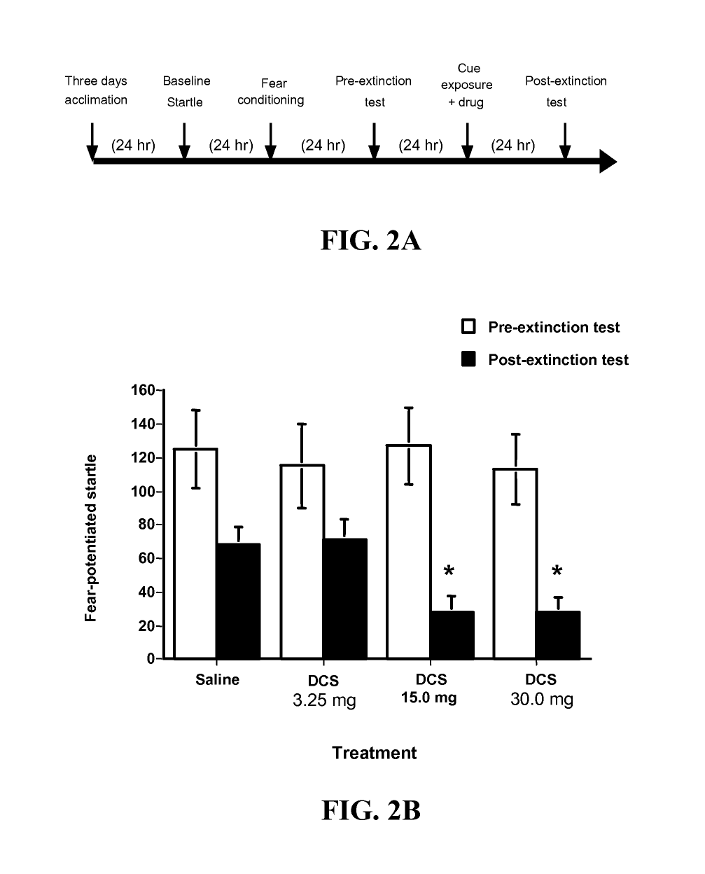

DCS facilitated extinction in a dose-dependent manner (FIG. 2B). ANOVA indicated a significant Dose effect, F(3,23)=3.02, with a significant linear trend, F(1,23)=7.26. Fear-potentiated startle was significantly lower in rats injected with 15 and 30 mg/kg DCS prior to extinction training, t(12)=2.61 and t(11)=2.53, for 15 and 30 mg/kg versus saline, respectively. Because 15 mg/kg produced the maximal enhancing effect, we used this dose in our subsequent experiments.

### Experiment 3

### Effect of DCS in Non-Extinguished Rats

To test whether the effects of DCS reflected an augmentation of extinction per se, or reflected, instead, a disruption of fear-potentiated startle independent of extinction (e.g., a delayed effect on the expression of fear-potentiated startle 24 hours after drug administration), additional rats were tested with and without extinction training. For this experiment, 28 rats were matched into 4 groups of 7 animals each based on the pre-test. 24 hrs later, each rat was injected with either saline or DCS (15 mg/kg) and returned to its home cage until placed in the startle chamber 30 min later. Two groups (one group of saline-injected rats and one group of DCS-injected rats) underwent extinction training. Two other groups (one group of saline-injected rats and one group of DCS-injected rats) were placed into the test chamber but did not receive extinction training. 24 hrs later, all groups were tested for fear-potentiated startle without drug injections.

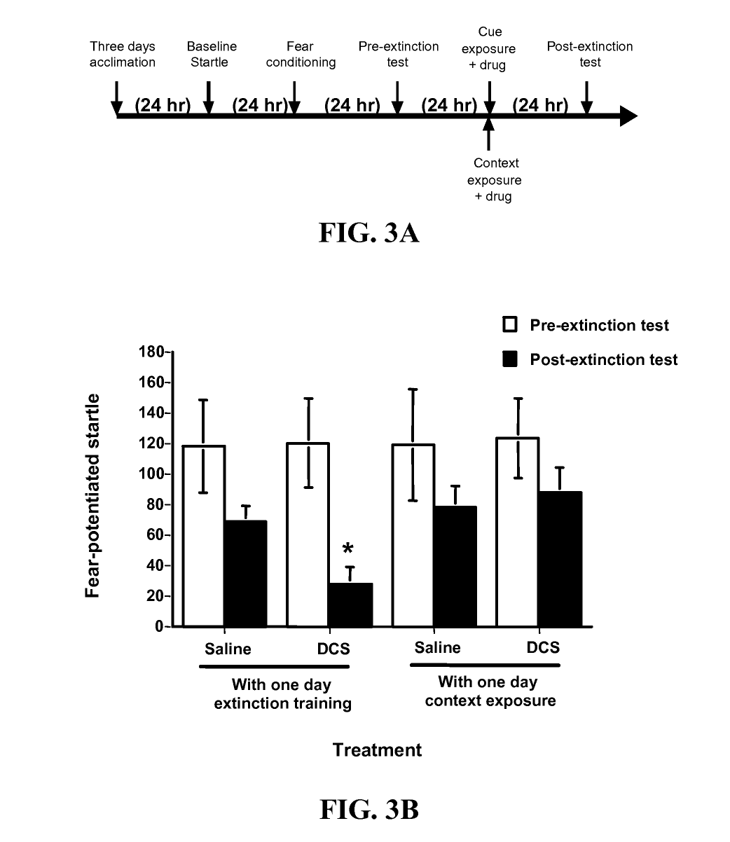

FIG. 3B shows that fear-potentiated startle in rats receiving DCS plus extinction training was significantly lower than in rats that received saline plus extinction training, t(12)=3.02. This replicates the principal finding of Experiment 2. The novel finding here is that fear-potentiated startle in rats that received DCS without extinction training was comparable to fear-potentiated startle in rats that received saline without extinction training. Thus, the effect of DCS noted in Experiment 2, and replicated here, appears to reflect a specific influence on extinction and not a more general effect on fear-potentiated startle measured 24 hours later in the absence of the drug.

### Experiment 4

### Effect of the Strychnine-Insensitive Glycine Recognition Site Antagonist, HA-966, on Extinction and on the Facilitation of Extinction by DCS

If DCS facilitates extinction by acting as an agonist at the strychnine-insensitive glycine recognition site, then the effect of DCS should be blocked by a strychnine-insensitive glycine site antagonist. To test this, 28 rats were matched into 4 groups of 7 animals each based on the pre-extinction test. 24 hrs later, each rat was injected with either saline or HA-966 (6 mg/kg) followed 10 min later by a second injection of either saline or DCS (15 mg/kg). This dose was chosen based on pilot experiments suggesting that higher doses of HA-966 alone blocked extinction, thereby complicating interpretations of interactive DCS/HA-966 effects. 30 min later, rats received a single session of extinction training and, 24 hrs later, were tested for fear-potentiated startle with no drug injections.

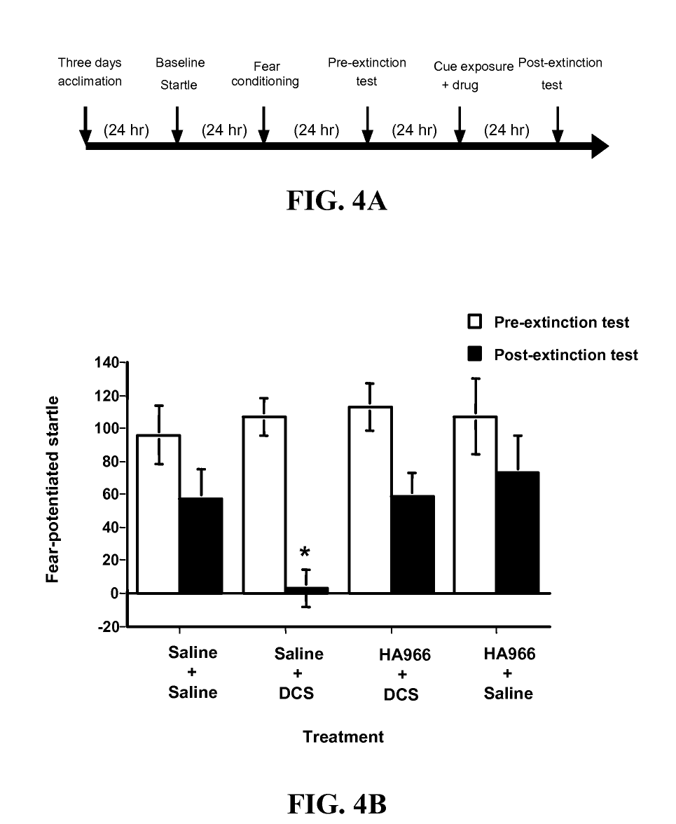

HA-966 completely blocked the enhancement of extinction produced by DCS, but did not itself influence extinction when administered alone (FIG. 4B). Replicating findings from experiments 2 and 3, fear-potentiated startle was significantly lower in rats injected with saline+DCS compared to rats injected with saline+saline, t(12)=2.73. This effect was blocked by HA-966. Fear-potentiated startle in rats injected with HA-966+DCS was not significantly different from fear-potentiated startle in rats injected with saline+saline, but was significantly different from fear-potentiated startle in rats injected with saline+DCS, t(12)=3.35. Overall, these results suggest that the facilitatory effect of DCS on extinction is most likely mediated by the NMDA receptor.

### Experiment 5

### Effect of Pre-Test DCS and HA-966 Administration on Fear-Potentiated Startle

This experiment evaluated whether the effect of DCS or HA-966 might be secondary to effects on fear itself or on CS processing. For example, if DCS increases CS-elicited fear, this might facilitate extinction by increasing the discrepancy between what the CS predicts and what actually occurs (Wagner and Rescorla (1972) “Inhibition in Pavlovian Conditioning: Application of a Theory,” in Inhibition and Learn., eds. Boakes and Halliday (Academic Press, London)). If HA-966 interferes with visual processing, this might block extinction produced by non-reinforced exposures to the visual CS. To evaluate these possibilities, 17 rats (Saline, N=5; DCS, N=6; HA-966, N=6) were acclimated, tested for baseline startle, and fear-conditioned as previously described. 24 hrs later, rats were injected with saline, DCS (15 mg/kg), or HA-966 (6 mg/kg). 30 (for DCS) or 40 (for HA-966) minutes after the injections, rats were tested for fear-potentiated startle.

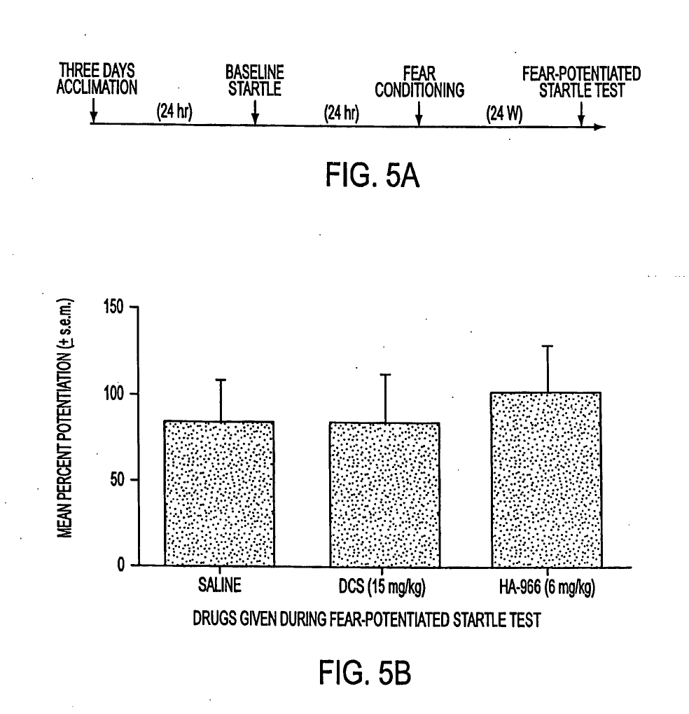

As shown in FIG. 5B, neither DCS nor HA-966 significantly influenced fear-potentiated startle when injected prior to testing. Thus, it is unlikely that these compounds influence extinction by increasing fear or by disrupting CS processing. In fact, a previous study reported a modest anxiolytic effect of both compounds on fear-potentiated startle (Anthony and Nevins (1993) Eur. J. Pharmacol. 250:317-324), although at doses higher than those used in the present study. Anxiolytic effects of DCS have also been reported with the elevated plus-maze (Karcz-Kubicha et al. (1997) Neuropharmacol. 36:1355-1367) and, at very high doses, with the Vogel-conflict procedure (Klodzinska and Chojnacka-Wojcik (2000) Psychopharmacologia 152:224-228).

### Experiment 6

### Effect of Intra-Amygdala DCS Infusions on Extinction

Previous studies indicate that NMDA receptors in the amygdala play a critical role in the extinction of conditioned fear (Falls et al. (1992) J. Neuroscience 12:854-863; Lee and Kim (1998) 1 Neuroscience 18:8444-8454). It is possible that the effect of systemically administered DCS reported in the above experiments was mediated by actions at amygdala NMDA receptors. To determine if the effect of systemically administered DCS would be mimicked by intra-amygdala DCS infusions, 36 rats with intra-amygdala cannulations received fear conditioning, extinction training, and testing for fear-potentiated startle as previously described. 15 minutes before being placed into the test chamber for extinction training, rats were infused with either phosphate-buffered saline (PBS) or DCS (10 μg/side) (preliminary findings suggested a weak effect of 1 μg/side and a more potent effect of 10 μg/side). One group of PBS-infused rats and one group of DCS-infused rats received extinction training. An additional group of PBS- and an additional group of DCS-infused rats were not placed in the test chamber and did not receive extinction training. Note that this procedure differed from that of Experiment 3 in which control rats received context exposure. Because context exposure constitutes context extinction, and because we were particularly concerned in this experiment that intra-amygdala DCS infusions might be associated with neurotoxicity, we wanted to ensure that any loss of fear-potentiated startle following intra-amygdala infusions could unambiguously be attributed to amygdala damage. If, for example, control rats that had received context extinction showed a reduction of CS-elicited fear, it would be unclear if this was attributable to a DCS-induced lesion or due, instead, to an unintended effect of context extinction on fear to the visual CS. Rats in all groups were tested 24 hours later without drug infusions.

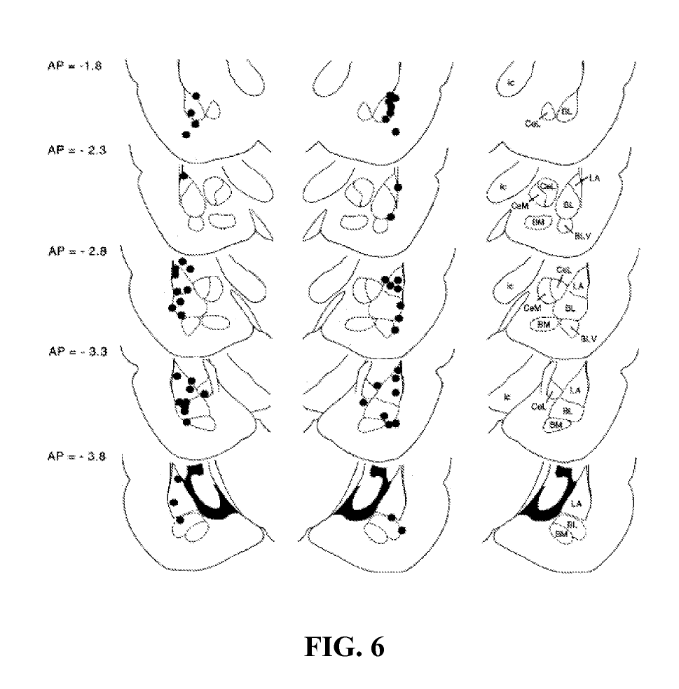

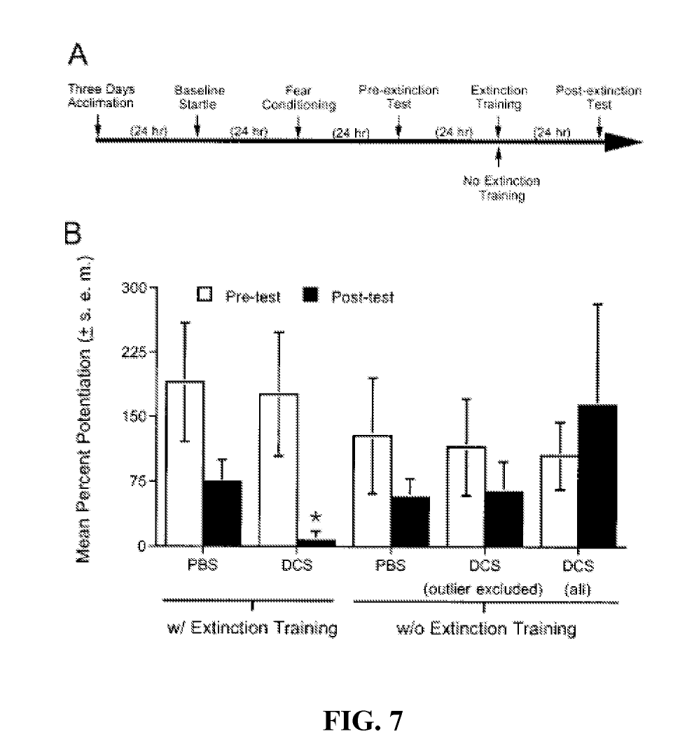

Behavioral data for 10 rats were excluded because the placements for these rats were located outside of the amygdala, resulting in group N's of 9 (PBS—extinction), 9 (DCS—extinction), 4 (PBS—no extinction), and 4 (DCS—no extinction). Placements for the remaining rats are shown in FIG. 6, and the behavioral results are shown in FIG. 7. ANOVA indicated a significant Treatment (DCS versus PBS) X Training (extinction versus no extinction) interaction, F(1, 22)=5.05. Fear-potentiated startle was significantly lower in rats that received intra-amygdala DCS infusions prior to extinction training compared to rats that received intra-amygdala PBS infusions prior to extinction training, t(16)=2.49, and was also significantly lower than in rats that received DCS without extinction training, t(11)=2.36. Fear-potentiated startle was not significantly different in rats that received PBS versus DCS infusions and no extinction training. The latter result suggests that the effect of DCS in rats that received extinction training is not attributable to neurotoxic DCS effects insofar as this would have disrupted fear-potentiated startle in both groups. In fact, fear-potentiated startle was unusually high in non-extinguished rats that received DCS infusions. This was largely attributable to a single rat with a percent increase score of 465%. Even with this outlier excluded, fear-potentiated startle was not significantly different in rats that received PBS versus DCS infusions and no extinction training. As before, however, fear-potentiated startled was significantly lower in rats that received intra-amygdala DCS infusions prior to extinction training compared to rats that received intra-amygdala DCS infusions without extinction training, 410)=2.34.

### Effects of DCS and HA-966 on Extinction Are Not Due to Changes in Baseline Startle

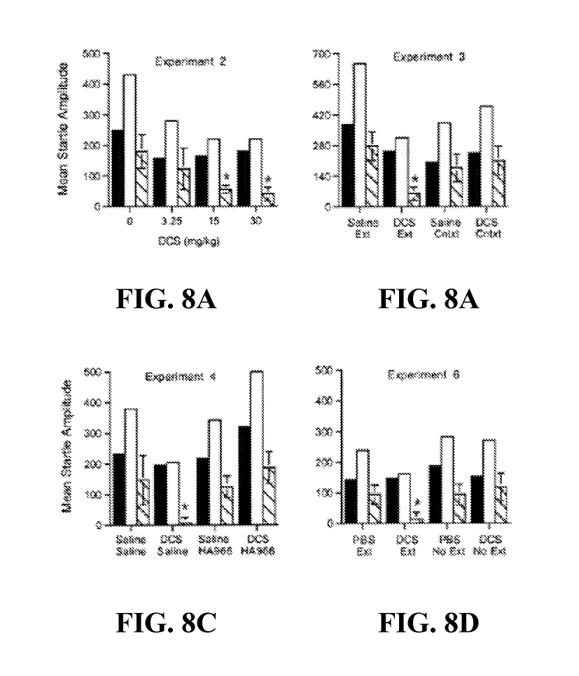

FIG. 8 shows absolute startle values from Experiments 2, 3, 4, and 6 (all experiments showing drug effects on extinction). Significant drug effects on baseline startle were not found in any experiment when measured in the extinction test 24 hours later. Moreover, the statistical results from analyses of percent potentiation scores were mostly comparable to results obtained using absolute difference scores. Thus, DCS dose-dependently facilitated extinction, F(1,24)=6.03 (Experiment 2). Fear-potentiated startle in the DCS+extinction group was significantly different from fear-potentiated startle in the saline+extinction group in Experiment 3, t(12)=3.21, and fear-potentiated startle was comparable in saline and DCS groups that did not receive extinction training. The difference between fear-potentiated startle in DCS+saline injected versus DCS+HA-966 injected rats approached but did not reach significance, t(12)=1.86, p=0.087 (Experiment 4). Also, fear-potentiated startle was significantly lower in rats that received intra-amygdala DCS infusions prior to extinction training compared to rats that received PBS infusions, t(16)=2.24 (Experiment 6).

### Discussion

### Experiments 1-6

The primary finding of these experiments is that DCS, a partial agonist at the strychnine-insensitive glycine-recognition site on the NMDA receptor complex, facilitates extinction of conditioned fear following either systemic injections (Experiments 2, 3, and 4) or intra-amygdala infusions (Experiment 6). Because DCS reduced fear-potentiated startle only in rats that concurrently received extinction training (Experiments 3 and 6), the effects of DCS cannot readily be attributed either to DCS-related neurotoxicity or to anxiolytic drug actions still present 24 hours after drug administration (i.e., during testing). The blockade of DCS's facilitatory influence on extinction by the glycine recognition site antagonist, HA-966, strongly suggests that the effect of DCS was mediated by interactions with the NMDA receptor (Experiment 4). This seems particularly likely insofar as the dose of HA-966 used did not, on it's own, increase fear-potentiated startle. Thus, the ability of HA-966 to reverse DCS effects on extinction cannot be attributed to a summation of independent facilitatory and disruptive effects, mediated by actions on different systems. The failure of either compound to influence fear-potentiated startle when given prior to testing suggests that their effects on extinction reflect direct effects on learning processes rather than on CS-processing or on fear itself.

As indicated earlier, extinction is generally thought to reflect the formation of new inhibitory associations, as opposed to the forgetting of previously formed associations (Pavlov (1927) Conditioned Reflexes (University Press, Oxford); Konorski (1948) Conditioned Reflexes and Neuronal Organization (University Press, London, Cambridge); Bouton and Bolles (1985) Context, Event Memories, and Extinction (Lawrence Erlbaum Associates, Hillsdale, N.J.); Falls and Davis (1995) “Behavioral and Physiological Analysis of Fear Inhibition,” in Neurobiological and Clinical Consequences of Stress: From Normal Adaptation to PTSD, eds. Friedman et al. (Lippincott-Raven Publishers, Philadelphia); Davis et al. (2000) “Neural Systems Involved in Fear Inhibition: Extinction and Conditioned Inhibition,” in Contemporary Issues in Modeling Psychopathology, eds. Myslobodsky and Weiner (Kluwer Academic Publishers, Boston); Rescorla (2001) “Experimental Extinction,” in Handbook of Contemporary Learning Theories, eds. Mowrer and Klein (Erlbaum, Mahwah, N.J.)). Consistent with this view, the evidence to date suggests that the neural mechanisms, neural circuitry, and pharmacology of excitatory fear conditioning and of conditioned fear extinction are similar. For example, systemic administration of the mitogen-activated protein kinase (MAPK) inhibitor, PD98059, as well as intra-amygdala PD98059 infusions, disrupt fear-conditioning as assessed with both freezing (Schafe et al. (2000) J. Neuroscience 20:8177-8187) and shock-motivated avoidance learning (Walz et al. (1999) Behav. Pharmacol. 10:723-730; Walz et al. (2000) Neurobiol. Learn. Mem. 73:11-20) respectively, and intra-amygdala PD98059 infusions also disrupt extinction as assessed with fear-potentiated startle (Lu et al. (2001) J. Neuroscience 21:RC162). As previously noted, intra-amygdala AP5 infusions also block fear conditioning as assessed with either fear-potentiated startle or freezing and also block extinction in these same paradigms (Miserendino et al. (1990) Nature 345:716-718; Falls et al. (1992) J Neuroscience 12:854-863; Fanselow and Kim (1994) Behav. Neuroscience 108:210-212; Maren et al. (1996) Behav. Neuroscience 110:1365-1374; Lee and Kim (1998) J. Neuroscience 18:8444-8454; Walker and Davis (2000) Behav. Neuroscience 114:1019-1033).

Although DCS has previously been shown to enhance learning in a variety of learning paradigms (Monahan et al. (1989) Pharmacol. Biochem. Behav. 34:649-653; Flood et al. (1992) Eur. J. Pharmacol. 221:249-254; Thompson et al. (1992) Nature 359:638-641; Quartermain et al. (1994) Eur. J. Pharmacol. 257:7-12; Pitkanen et al. (1995) Eur. Neuropsychopharmacol. 5:457-463; Matsuoka and Aigner (1996) J. Pharmacol. Exp. Ther. 278:891-897; Pussinen et al. (1997) Neurobiol. Learn. Mem. 67:69-74; Land and Riccio (1999) Neurobiol. Learn. Mem. 72:158-168), this appears to be the first demonstration of an enhancement of extinction learning by DCS. In fact, Port and Seybold ((1998) Physiol. Behav. 64:391-393) reported that DCS retarded extinction of an appetitive instrumental response, and that the NMDA receptor antagonist MK801 enhanced extinction. The latter finding is in contrast to several other results showing that NMDA receptor antagonists disrupt extinction (Falls et al. (1992) J. Neuroscience 12:854-863; Cox and Westbrook (1994) Quarterly J. Exper. Psych. 47B:187-210; Baker and Azorlosa (1996) Behav. Neuroscience 110:618-620; Kehoe et al. (1996) Psychobiology 24:127-135; Lee and Kim (1998) J. Neuroscience 18:8444-8454). The data used to evaluate extinction in Port and Seybold (1998) Physiol. Behav. 64:391-393) were collected while animals were still under the influence of DCS (i.e., within-session extinction), and it is possible that effects on performance obscured effects on extinction. It is also possible, though less likely, that the extinction of instrumental responses responds differently to NMDA receptor manipulations than does the extinction of classically conditioned responses.

Findings implicating amygdala NMDA receptors in both excitatory fear conditioning and conditioned fear extinction are of considerable theoretical interest. Evidence that the extinction of conditioned fear memories might be accelerated by NMDA receptor agonists is also of considerable clinical interest. Many believe that the neural circuitry mediating adaptive fear is closely related if not identical to the neural circuitry mediating clinical fear (e.g., in post-traumatic stress disorder; Rosen and Schulkin (1998) Psychological Review 105:325-350, 1998; Bouton et al. (2001) Psychological Review 108:4-32; Gorman et al. (2000) Am. J. Psychiatry 157:493-505). In clinical populations, a reduced ability to extinguish conditioned fear associations might contribute to the persistence of maladaptive fear and may reduce the effectiveness of therapeutic interventions that rely upon extinction processes (e.g., systematic desensitization, exposure, and imagery therapies). The results reported here suggest that the effectiveness of these traditional clinical approaches might be facilitated by pharmacological interventions that promote extinction. Clinical trials to test this idea are currently being planned.

### Experiment 7

### Clinical Trial of D-Cycloserine Augmentation of Behavioral Exposure Therapy for Specific Phobia [Formatting Change Only]

Experiment 7 outlines a proposed method for demonstrating the effect of D-cycloserine combined with psychotherapy, and Experiment 8 illustrates data utilizing a specific embodiment of this method. Acrophobia, or fear of heights, has been shown to be responsive to virtual reality exposure (VRE) therapy (Rothbaum et al. (1995) Am. J. Psychiatry 152(4):626-628), and VRE therapy has been well validated for different specific phobias and for post-traumatic stress disorder (Rothbaum et al. (1995) Am. J. Psychiatry 152(4):626-628; Rothbaum et al. (2000) J. Consult. Clin. Psych. 68(6):1020-1026). With VRE for fear of heights, it was shown that there were significant improvements on all outcome measures for the treated as compared to the untreated groups (Rothbaum et al. (1995) Am. J. Psychiatry 152(4):626-628). Treated participants in this study reported a positive attitude toward treatment, whereas untreated participants reported negative attitudes. VRE treatment for fear of flying demonstrated that VR treatment was equivalent to standard in vivo exposure therapy, both of which showed significant superiority to waitlist control on all outcome measures (Rothbaum et al. (2000) J. Consult. Clin. Psych. 68(6):1020-1026). In these studies, patients appear to improve steadily across sessions as noted by the decrease in subjective discomfort across sessions as would be expected with incremental habituation or extinction to the fearful stimulus.

In this experiment, acute treatment with an NMDA glutamate receptor agonist prior to psychotherapy is used to enhance the effects of VRE therapy. Specifically, an acute dose of D-Cycloserine (DCS) is given to a patient shortly before each individual therapy session over 2 weekly sessions to enhance the final level of VRE treatment efficacy.

### Dosing Rationale

DCS has been FDA approved for approximately 20 years, initially for the treatment of tuberculosis, and then as a cognitive enhancer in several clinical trials over the last decade. For tuberculosis, DCS is generally dosed at 500-1000 mg/day divided twice daily (PDR 1997) with chronic treatment. At a dose of 500 mg/day, blood levels of 25-30 mg/ml are generally maintained. The peak blood levels occur within 3-8 hours after dosing, and it is primarily renally excreted with a half-life of 10 hours. Infrequent side effects in patients on chronic dosing schedules (who were generally chronically ill with tuberculosis) include drowsiness, headache, confusion, tremor, vertigo, and memory difficulties, paresthesias, and seizure. Of note, no significant side effects have been reported in any of the clinical studies examining DCS for cognitive enhancement, even when used up to 500 mg/day doses (D'Souza et al. (2000) Biol. Psych. 47:450-462; Fakouhi et al. (1995) J. Geriatri. Psychiatry Neurol. 8(4):226-230; Randolph et al. (1994) Alzheimers Dis. Assoc. Disord. 8(3):198-205; van Berckel et al. (1996) Biol. Psychiatry 40(12):1298-1300).

In this experiment, a 50 mg or 500 mg dose of DCS is given to a patient acutely prior to psychotherapy for several reasons. The low dose is based on several clinical trials in which 30-100 mg/day given daily were effective for implicit memory (Schwartz et al. (1996) Neurology 46(2):420-424) and subscales of dementia rating in Alzheimer's disease (Tsai et al. (1999) Am. J. Psychiatry 156(3):467-469). Furthermore 50 mg/day appeared to be most effective in treatment of negative symptoms of Schizophrenia (Goff et al. (1996) Am. J. Psychiatry 153(12):1628-1630; Goff et al. (1999) Arch. Gen. Psych. 56(1):21-27). The higher dose (500 mg) is chosen because the efficacy of DCS in the lower dose range (10-250 mg/day) has not been effective in several trials by other groups (D'Souza et al. (2000) Biol. Psych. 47:450-462; Fakouhi et al. (1995) J. Geriatri. Psychiatry Neurol. 8(4):226-230; Randolph et al. (1994) Alzheimers Dis. Assoc. Disord. 8(3):198-205). Using Luteinizing Hormone (LH) secretion as a measure of NMDA receptor activation, it was shown that single doses of 15-150 mg of DCS did not lead to significant increases in LH (van Berckel et al. (1997) Neuropsychopharm. 16(5):317-324), but that a single 500 mg dose did effectively stimulate LH release (van Berckel et al. (1998) Psychopharm.138(2):190-197). At this dose, it was noted that there were no changes in cortisol, plasma HVA, or vital sign measures. Furthermore, at this dose there were no reported side effects and no changes in mood scores. Thus it was concluded that single doses as high as 500 mg in an otherwise drug naive, healthy individual would be well tolerated, without side effects, but with clear neuroendocrine effect (van Berckel et al. (1998) Psychopharm. 138(2):190-197).

The choice to use DCS in an acute treatment, rather than chronic, format is based on several factors. First is the novel and enormously useful clinical benefit that would be gained from a medication used in a time-limited fashion as an adjunct to psychotherapy. Second, and most importantly, is the issue that there may be significant compensatory changes in the NMDA receptor complex following chronic administration. As with all neurotransmitter receptors, regulation of the NMDA receptor is likely closely controlled for level of activity. Many chronically administered psychotropic agents are thought to function over a prolonged time due to the chronic downregulation of numerous receptor types (reviewed in Ressler and Nemeroff (1999) Biol. Psychiatry 46:1219-1233). Most of the extant preclinical data, on which the cognitive enhancement effect of DCS is based, are acute treatment studies in animals (Flood et al. (1992) Neurosci. Lett. 146:215-218; Land and Riccio (1999) Neurobiol. Learn. Mem. 72:158-168; Matsuoka and Aigner (1996) J. Pharmacol. Exp. Ther. 278:891-7). Several of the chronic treatment clinical trials have failed to show efficacy (D'Souza et al. (2000) Biol. Psych. 47:450-462; Fakouhi et al. (1995) J. Geriatri. Psychiatry Neurol. 8(4):226-230; Randolph et al. (1994) Alzheimers Dis. Assoc. Disord. 8(3):198-205). Direct studies in mice of acute versus chronic treatment with DCS suggest that chronic treatment does not enhance learning, whereas acute treatment clearly does (Quartermain et al. (1994) Eur. J. Pharmacol. 257(1-2):7-12). Furthermore, the relatively low side effect profile of DCS at chronic doses is almost negligible at acute doses, making acute treatment a safe and low-risk approach to treatment.

### Patient Selection

Although the majority of patients with fear of heights are expected to be simply phobic, it is expected that a substantial minority may be agoraphobic. In this experiment, a patient must meet DSM-IV criteria for specific phobia, situational type (i.e., fear of heights) or panic disorder with agoraphobia in which heights are the feared stimulus, or agoraphobia without a history of panic disorder, in which heights are the feared stimulus.

### Treatment Schedule

A patient is treated once per week for 2 weeks, with a 50 mg or 500 mg DCS dose administered only on the day of therapy, approximately 4 hours before the initiation of therapy. Thus a patient receives only two doses of medication or placebo total over the 2-week period.

Virtual reality exposure therapy (VRE) is to a series of footbridges over a canyon and a glass elevator that rises 49 floors (Rothbaum et al. (1995) Am. J. Psychiatry 152(4):626-628). During VRE sessions the patient wears a head-mounted display with stereo earphones that provides visual and audio cues consistent with being on a footbridge over a canyon or inside a glass elevator. During therapy, the therapist makes appropriate comments and encourages continued exposure until anxiety has habituated.

During each VRE session, anxiety is rated by subjective units of discomfort (SUDs) on a 0 to 100 scale in which 0 indicates no anxiety and 100 indicates panic-level anxiety. Psychophysiological responses (pulse, BP, GSR) are monitored throughout each exposure session.

### Assessment

A patient's response to a combination therapy session of DCS and VRE may be assessed using any of the methods listed below. Table 1 shows an assessment schedule for a patient done both before and after the combination therapy.

### Assessment Methods

### a) Interviews

The Initial Screening Questionnaire (Rothbaum et al. (1995) Am. Psychiatry 152(4):626-628) is a short screening instrument that is used to screen initial phone inquiries to identify those likely meeting study criteria for fear of heights.

The Structured Clinical Interview for the DSM-IV (Spitzer et al. (1987) Structured Clinical Interview for DSM III-R (SCID) (New York State Psychiatric Institute, Biometrics Research, New York)) is administered to diagnose and screen for various DSM-III-R axis I disorders (e.g., schizophrenia) as well as establish co-morbid diagnoses.

The Clinical Global Improvement (CGI) Scale is a global measure of change in severity of symptoms. The scale is bipolar with 1=very much improved; 7=very much worse; and 4=no change. It has been used extensively in clinical trials for a variety of psychiatric patients (Guy (1976) ECDEU Assessment Manual for Psychotherapy (revised ed., National Institute of Mental Health, Bethesda, Md.)).

### b) Self-report Measures

The Acrophobia Questionnaire (AQ) is a short self-report questionnaire assessing specific symptoms of fear of heights. It is given weekly prior to VRE.

The Attitude Towards Heights Questionnaire (ATHQ) is a separate self-report scale that measures slightly different aspects of avoidance, and other fear of heights related phenomena.

The Rating of Fear Questionnaire (RFQ) (Rothbaum et al. (1995) Am. J. Psychiatry 152(4):626-628) is used to further assess level of fear related to heights in general and the VRE therapy.

The State-Trait Anxiety Inventory (STAI; Spielberger et al. (1970) Manual for the State-Trait Anxiety Inventory (self-evaluation questionnaire) (Consulting Psychologists Press, Palo Alto, Calif.)) is comprised of 40 items divided evenly between state anxiety and trait anxiety. The authors reported reliability for trait anxiety was 0.81; as expected, figures were lower for state anxiety (0.40). Internal consistency ranges between 0.83 and 0.92.

The Beck Depression Inventory (BDI; Beck et al. (1961) Archives of Gen. Psych. 4:561-571) is a 21-item self-report questionnaire assessing numerous symptoms of depression. The authors report excellent split-half reliability (0.93), and correlations with clinician ratings of depression range between 0.62 and 0.66.

### c) Therapist Measure

The subjective units of discomfort (SUDs) is scored by the therapist based on the participant's report during the VRE at 5 minute intervals. SUDS are rated on a 0 to 100 scale in which 0 indicates no anxiety and 100 indicates panic-level anxiety

The Behavioral Avoidance Test (BAT) consists of a brief re-exposure to heights via the Virtual Reality environment, in which the therapist assesses the patients subjective level of fear and avoidance of heights.

### d) Psychophysiological Measures

Measurement of heart rate (HR) is performed and stored by a non-invasive, computer controlled monitoring device for assessment of autonomic reactivity during VRE.

Measurement of blood pressure (BP) is performed by a non-invasive, computer controlled sphygmomanometer for assessment of vascular tone and autonomic reactivity during VRE

Measurement of galvanic skin conductance (GSR) is performed by a non-invasive, computer controlled monitoring device for assessment of autonomic fear responsivity during VRE.

TABLE 1

| Assessment Session | Measures |
|---|---|
| Prior to entry | Consent form |
| | SCID |
| Pre-treatment Assessment | Acrophobia Questionnaire |
| | Attitude Towards Heights Questionnaire |
| | Ratings of Fear Questionnaire |
| | Behavioral Avoidance Test |
| | BDI |
| | STAI |
| Weekly VRE Therapy | Psychophysiologic measures (HR, BP, GSR) |
| Sessions (×2) | SUDs |
| Post-VRE Assessments | Acrophobia Questionnaire |
| and 6 Month Follow up | Attitude Towards Heights Questionnaire |
| Assessment | Ratings of Fear Questionnaire |
| | CGI |
| | Behavioral Avoidance Test |

### Future Directions

The results presented herein demonstrate that a pharmacologic agent that enhances extinction learning can be administered acutely in combination with a session of psychotherapy, thereby enhancing the effectiveness of the psychotherapy session. The present invention contemplates a variety of specific parameters for such a combination therapy protocol, including the choice of psychotherapy used, the psychiatric disorders to be treated, the particular pharmacologic agent to be used in the methods of the invention, and the timing and dosage of administration of the pharmacologic agent. Particular manifestations of these parameters as contemplated in the present invention are discussed in more detail in the foregoing detailed description of the invention.

### Experiment 8

### Clinical Trial of D-Cycloserine Augmentation of Behavioral Exposure Therapy for Specific Phobia

The methods described in Example 7 predict that the effectiveness of a psychotherapy session can be enhanced via acute administration of a pharmacologic agent that enhances extinction learning in combination with the session of psychotherapy. The present example provides results from an experiment based on the methods described in Example 7.

The research protocol used in this study was approved by the Emory University Institutional Review Board and all subjects gave written informed consent for participation in the study.

Patient Selection and Group Assignment

Twenty-eight volunteer patients (or participants) were recruited from the general community with no currently active psychiatric disorders except for acrophobia. The diagnosis of acrophobia (a subtype of Specific Phobia), requires an excessive or unreasonable fear of heights that interferes significantly with the person's normal routine and functioning, and is characterized by severe anxiety in the presence of height situations.

One participant did not return after the pre-assessment. Therefore, 27 patients (11 male, 16 female) were randomly assigned to three treatment groups via a predetermined and blinded order of treatment assignment. These groups were: 1) Placebo+VRE Therapy (n=10); 2) 50 mg D-cycloserine+VRE Therapy (n=8); and 3) 500 mg D-cycloserine+VRE Therapy (n=9). Treatment condition was double-blinded, such that the subjects, therapists, and assessors were not aware of assigned study medication condition. The blind was maintained throughout the duration of the study. All 27 patients completed pretreatment, both therapy sessions, and the 3-month follow-up assessment. Pre-treatment data are listed in Table 2.

TABLE 2

| Characteristic | Placebo (n = 10) | D-Cycloserine (N = 17) | p value |
|---|---|---|---|
| Age | 44.8 ± 2.3 | 46.4 ± 2.8 | 0.68 |
| DSM-IV Dx | 2.1 ± .69 | 1.6 ± .24 | 0.41 |
| GAF | 64.7 ± 1.3 | 65.1 ± .72 | 0.76 |
| BDI | 7.7 ± 4.4 | 4.2 ± 1.1 | 0.34 |
| STAT-state | 34.2 ± 5.6 | 33.9 ± 2.7 | 0.96 |
| STAT-trait | 31.7 ± 4.5 | 31.4 ± 1.9 | 0.95 |
| AAQ | 65.8 ± 6.2 | 73.4 ± 5.6 | 0.39 |
| AAVQ | 18.7 ± 2.7 | 24.2 ± 2.5 | 0.17 |
| ATH | 54.4 ± 1.7 | 53.9 ± 1.7 | 0.84 |

Abbreviations:
DSM-IV DX, number of DSM-IV diagnoses by SCID; GAF, Global Assessment of Functioning Scale; BDI, Beck Depression Inventory; STAI, state-trait anxiety scale; AAQ, acrophobia anxiety questionnaire; AAVQ, acrophobia avoidance questionnaire; ATH, attitude towards heights inventory.
Values are given as mean ± sem.

Patient Assessment

Patients were examined with a battery of screening tests.

Acrophobia and other psychiatric diagnoses were determined by interview with the Structured Clinical Interview for DSM-III-R (Spitzer R L, Williams J B, Gibbon M, & First MB. The Structured Clinical Interview for DSM-III-R (SCID): I. History, rationale, and description. Archives of General Psychiatry 1992; 49:624-629).

The Acrophobia Questionnaire with Avoidance (AAVQ) and Anxiety (AAQ) subscales were used to examine their fear of heights (Cohen D. Comparison of self-report and overt-behavioral procedures for assessing acrophobia. Behav Therapy 1977; 8:17-23).

The Attitudes Towards Heights Inventory (ATHI) was used to examine their fear of heights (Rothbaum B O, Hodges L F, Kooper R, Opdyke D, Williford JS, & North M. Effectiveness of computer-generated (virtual reality) graded exposure in the treatment of acrophobia. Am J Psychiatry 1995; 152:626-8; Abelson J & Curtis G. Cardiac and neuroendocrine responses to exposure therapy in height phobics: desynchrony within the “physiological response system.” Behav Res Ther 1989; 27:561-567).

The Beck Depression Inventory (BDI) and the State/Trait Anxiety Inventory (STAI) were used to examine their general levels of depression and anxiety (Beck A, Ward C, Mendelsohn M, Mock J, Erbaugh J. An inventory for measuring depression. Arch Gen Psych 1961; 4:561-571; Spielberger C, Gorsuch R, Lushene R. Manual for the State-Trait Anxiety Inventory (self-evaluation Questionnaire). Palo Alto: Consulting Psychologists Press; 1970).

Overall global improvement was assessed with the Clinical Global Improvement (I) Scale (CGI). During the initial screen, patients also had limited but structured exposure to the virtual reality height environment during a Behavioral Avoidance Test (BAT), in which they reported on a 0-100 scale (100 being the most intense fear) their subjective units of discomfort (SUDS) for each floor (floors 1-19) of the virtual glass elevator.

Electrodermal skin conductance fluctuations (SCF) were measured as described previously (Grillon C & Hill J. Emotional arousal does not affect delay eyeblink conditioning. Cogn Br Research 2003; 17: 400-405; Storm H, Myre K, Rostrup M, Stokland O, Lien M D, & Raeder J C. Skin conductance correlates with perioperative stress. Acta Anaesthesiol Scand 2002; 46:887-95; Lader M H. Palmar skin conductance measures in anxiety and phobic states. J Psychosom Res 1967; 11:271-281. Finger electrodes (ProComp Module, Thought Technology Ltd., Montreal) were worn by the subject during the initial and post-treatment behavioral assessment tests. Data are reported as number of SCFs per minute of exposure. SCF was measured as in Grillon and Hill using fluctuation defined as 0.05 μS deviation in baseline skin conductance (Grillon C & Hill J. Emotional arousal does not affect delay eyeblink conditioning. Cogn Br Research 2003; 17: 400-405). SCF was averaged over the entire exposure and presented as fluctuations per minute. Each fluctuation was defined as ≧2 second deviation of 0.05 μS from the local mean (average baseline +/−30 seconds). Follow-up analyses also examined fluctuations as defined by a ≧2 second deviation of 5% greater or less than the local mean.

Medication

D-cycloserine (Seromycin, 250 mg, Eli Lilly Pharmaceuticals) was reformulated into 50 mg or 500 mg with identical placebo capsules.

No adverse events occurred during the study. Although reports of side effects were not systematically obtained, subjects were routinely asked if they were experiencing any difficulties. Upon breaking the blind no difference was found between subjects reporting side effects with placebo or D-cycloserine.

Treatment Schedule

With VRE for fear of heights, a virtual glass elevator was used in which patients stood while wearing a VRE helmet and were able to peer over a virtual railing. Computerized effects give a real sense of increase in height as the elevator rises. Previous work has shown improvements on all acrophobia outcome measures for treated as compared to untreated groups after 7 weekly, 35-45 min therapy sessions (Rothbaum B O, Hodges L F, Kooper R, Opdyke D, Williford J S, & North M. Effectiveness of computer-generated (virtual reality) graded exposure in the treatment of acrophobia. Am J Psychiatry 1995; 152:626-8).

Patients underwent two 35-45 min therapy sessions, which is a suboptimal amount of exposure therapy for acrophobia (Rothbaum B O, Hodges L F, Kooper R, Opdyke D, Williford J S, & North M. Effectiveness of computer-generated (virtual reality) graded exposure in the treatment of acrophobia. Am J Psychiatry 1995; 152:626-8). These two therapy sessions were separated by 1-2 weeks (average=12.9 days). Patients were instructed to take a single pill of study medication (placebo, D-cycloserine 50 mg, or D-cycloserine 500 mg) 2-4 hours before each therapy session, such that only two pills were taken for the entire study. There were no adverse events reported from either group taking placebo or drug prior to exposure therapy.

A mid-treatment assessment occurred 1 week after the first treatment (average=7.2 days), a post-treatment assessment was performed 1-2 weeks following the final therapy session (average=11.5 days), and an additional follow-up assessment was performed 3 months after the therapy (average=107.5 days).

Assessment

Patients, therapists and assessors were kept blind to treatment condition throughout the study. All data were entered into the SPSS statistics package by research assistants also blind to condition. Pre-treatment variables (Table 2) were analyzed using t-tests for independent samples. Post-treatment variables (skin conductance fluctuations, AAQ, AAVQ, ATH, CGI, and number of self exposure to heights) were analyzed using one-way ANOVA or Repeated Measures ANOVA with time and drug condition as separate factors.

Specific comparisons of different floors and SUDS within treatment sessions were performed with one-way ANOVA with the between-subjects factor of drug vs. placebo group. The effect of interaction between drug group and different floors or drug group and different time points on the SUDS score (FIG. 1) was performed using multivariate analysis with repeated measures with floor or time as the repeated within-subjects factor and drug condition the independent between-subjects factor. The effect of these interactions on SUDS as the outcome variable for the pre-post analysis (FIG. 2) was performed with an overall ANOVA with pre-post difference and floor as within-subjects factor and drug group as between-subjects factor.

Results

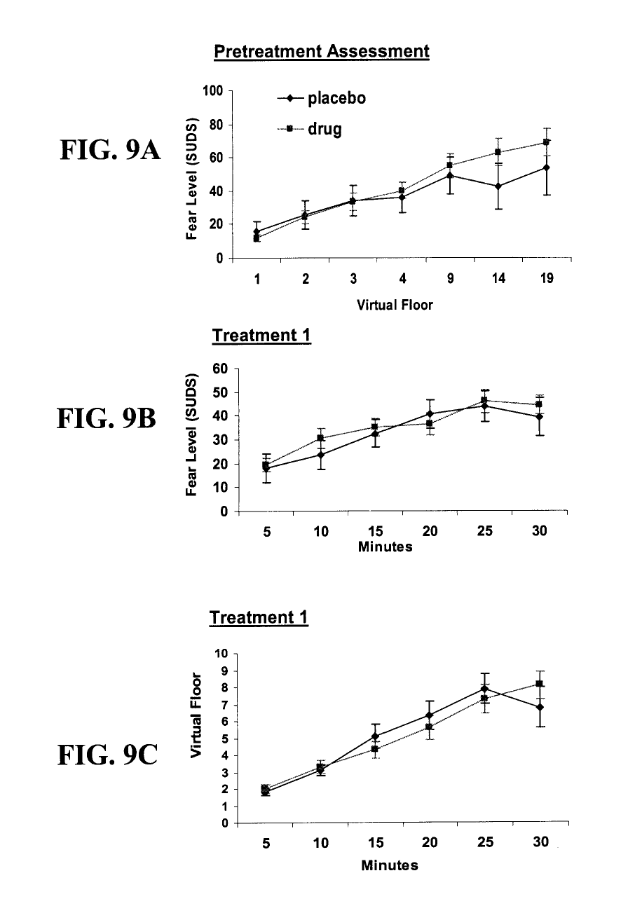

Twenty-seven patients completed the two therapy sessions, with ten subjects randomly assigned to placebo (5 m, 5 f), and seventeen subjects randomly assigned to D-cycloserine (6 m, 11 f). At the pretest assessment there was no difference in age, number of DSM-IV diagnoses, global assessment of functioning (GAF), or scores on the BDI, STAI-state, or STAI-trait between placebo and drug groups (Table 2). There was also no difference in initial acrophobia measures (Table 2) or in SUDS levels at different floors within the virtual elevator environment (FIG. 9A).

Following treatment, statistically significant differences were found between placebo and drug groups for almost every primary outcome measure. In the results below, statistics are presented for ANOVA measures with the drug groups both separated and combined. Analysis of the data indicated that there were no significant differences between the 50 mg and 500 mg drug groups for the primary outcome measures of acrophobia (ANOVA, p's>0.5); therefore the data in the figures are presented with drug groups combined.

D-Cycloserine does not Affect Baseline Level of Fear

Because no direct anxiolytic effect of D-cycloserine was anticipated based on preclinical studies and also because there was no retention interval to allow facilitative effects of D-cycloserine on extinction learning, no effects of D-cycloserine were anticipated for session one. Consistent with this, no differences were found between groups in SUDS level during the first therapy session (FIG. 9B). During the therapy sessions, patients have some control over how high the elevator is allowed to rise, permitting an analysis of avoidance of heights. During this first session, no differences were found in the highest floor attained at different time points (FIG. 9C). These findings indicate that the presence of D-cycloserine during the therapy session did not affect level of fear or avoidance of fear during the therapy.

D-Cycloserine Enhances Extinction of Fear within the Virtual Environment

During the second session, patients in the D-cycloserine group experienced lower SUDS than the placebo group (SUDS at 5 minutes, F(1,25)=7.1, p≦0.01), and they elevated to higher floors after 20 minutes, (mean floor for placebo=13.0, D-cycloserine=15.9, F(1,25)=6.3; p≦0.01). This suggests that during the second session, there was less fear and avoidance in the group that had received D-cycloserine during the first session. The D-cycloserine group also showed more improvement as measured by participant scores on the CGI scale at the second session (placebo=2.8 vs D-cycloserine=2.25; F(1,25)=5.2; p≦0.05).

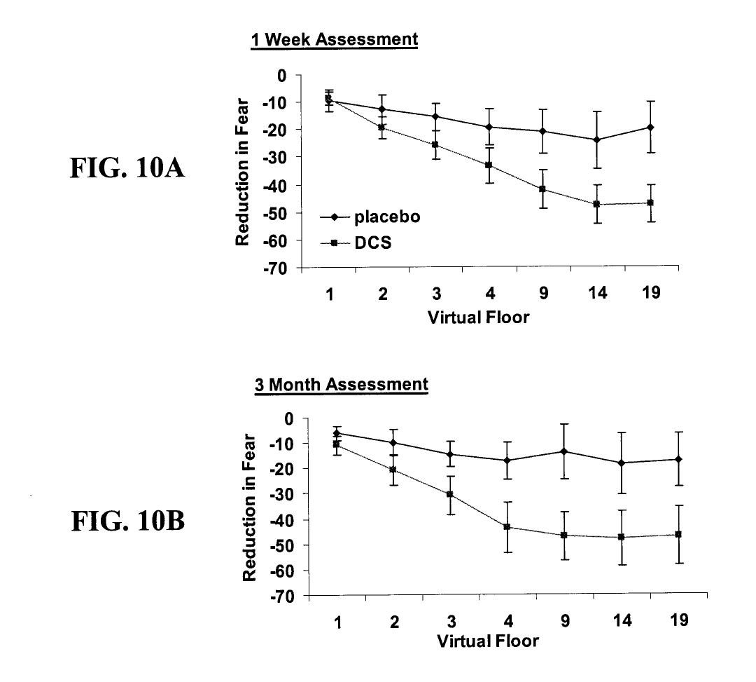

One week after the second session a post-treatment assessment was performed in the absence of drug and the difference scores between the post-treatment and pre-treatment were examined. The group that received D-cycloserine during the therapy sessions showed significantly less fear of heights as determined by SUDS at successive elevator floors during the BAT VR assessments (FIG. 10A; F(6,150)=3.8, p≦0.001). This difference was also seen if the two separate doses of drug were analyzed separately with a repeated measures ANOVA (F(12, 144)=2.7, p≦0.01). The continued decrease in fear within the virtual environment in the absence of D-cycloserine demonstrates that, as in animal experiments, the enhancement of extinction in humans with D-cycloserine is not state-dependent (Walker D L, Ressler K J, Lu K T & Davis M. Facilitation of conditioned fear extinction by systemic administration or intra-amygdala infusions of D-cycloserine as assessed with fear-potentiated startle in rats. J Neurosci 2002; 22:2343-51; Ledgerwood L, Richardson R, & Cranney J. Effects of D-cycloserine on extinction of conditioned freezing. Behav Neurosci 2003; 117:341-9). These data suggest that two sessions of VRE therapy in combination with D-cycloserine for fear of heights is sufficient for extinction of fear within the virtual environment (FIG. 10A).

Enhanced Extinction with D-Cycloserine is Maintained at 3 Months

To evaluate how D-cycloserine would affect retention of extinction, as well as whether it would generalize to real life situations outside the VR environment over time, subjects were asked to return for a follow-up session 3 months after their VRE treatment. Twenty-one of the 27 completing patients returned for follow-up assessment [8 placebo (80% of enrolled), 13 D-cycloserine (77% of enrolled)]. Analysis of the pre-treatment data and the one-week post-treatment assessments showed that there were no significant pre- or post-treatment differences on anxiety or fear measures between those that returned for follow-up and the six that did not.

At the follow-up assessment, subjects were tested again in the absence of D-cycloserine for their level of fear in the virtual elevator environment with the BAT. It was found that patients who received D-cycloserine maintained the specific decrease in fear to the virtual environment over the 3-month period as determined by SUDS during the exposure to virtual heights (FIG. 10B; F(6,102)=2.4, p≦0.05). No significant differences between the two different drug doses were found. This suggests that the extinction of fear that was enhanced in the drug group during the two therapy sessions was relatively robust and lasting.

Physiological Arousal and Fearfulness During Virtual Exposure

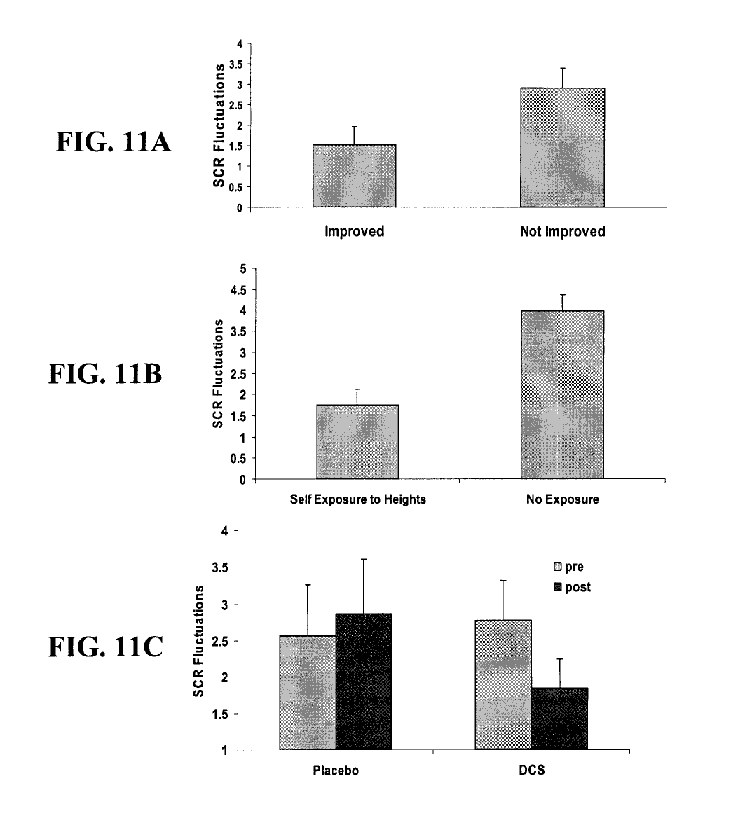

The number of spontaneous fluctuations of skin conductance is a common measure of emotional arousal and anxiety, such that those with more fear or anxiety typically show more spontaneous reactivity or fluctuation in their baseline skin conductance during provocation (Grillon C & Hill J. Emotional arousal does not affect delay eyeblink conditioning. Cogn Br Research 2003; 17:400-405; Lader M H. Palmar skin conductance measures in anxiety and phobic states. J Psychosom Res 1967; 11:271-281). Consistent with this, during the post-treatment behavioral assessment tests it was found that the number of spontaneous fluctuations correlated with the measures of subjective improvement in fear of heights. Those reporting “much” or “very much” improvement at the initial post-treatment assessment test showed significantly fewer spontaneous fluctuations than did those who reported no improvement or worsening (FIG. 11A; F(1,19)=4.5, p≦0.05; linear regression, r=0.44). Additionally, those who showed less avoidance of heights in the real world since treatment, as indicated by increased likelihood of exposing themselves to real-world heights, also showed fewer spontaneous fluctuations than did those who did not self-expose since treatment (FIG. 11B; F(1,19)=8.26; p≦0.01; linear regression, r=0.55).

It was also found that those subjects given D-cycloserine during exposure therapy had a significant decrease in average spontaneous fluctuations from pre- to post-treatment (FIG. 11C; paired t-test, p≦0.05) compared to those given placebo during the treatment (p≧0.5). Subsequent analysis of skin conductance fluctuations using the criterion of a 5% change from baseline in skin conductance instead of an absolute 0.05 μS difference also demonstrated a significant time X treatment effect (repeated measure ANOVA, F(1,19)=8.0, p≦0.01). These data suggest that the improvement in extinction of fear achieved with D-cycloserine augmentation during exposure was evident in both subjective and objective physiological measures of fear.

D-Cycloserine Augments Reduction of General Measures of Acrophobia

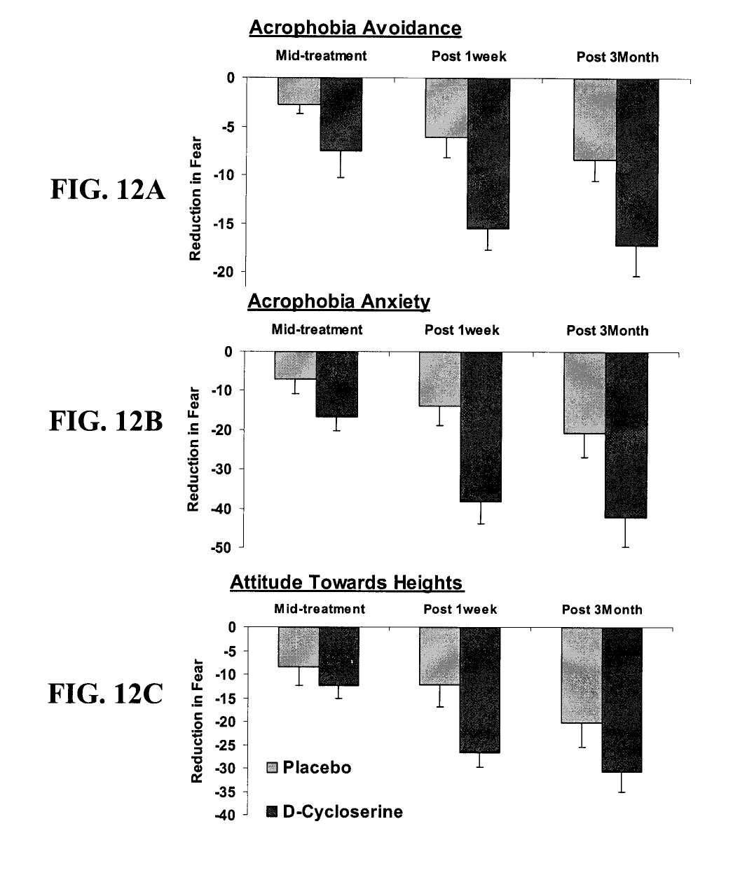

To examine the ability of the virtual reality exposure to heights to reduce symptoms of acrophobia in the real world, standard outcome measures of acrophobia that are not specific to the virtual environment were utilized. These measures were taken at the pretreatment assessment, the mid-treatment assessment between the two therapy sessions, 1-2 weeks post-treatment, and 3 months post-treatment. These measures were always taken in the absence of medication, and the questionnaires referred to subjects' symptoms of acrophobia in the real world, not the virtual environment. FIG. 12 shows the reduction of fear as measured by difference scores between each post-treatment measure and the pretreatment baseline measure for placebo and D-cycloserine groups.

For all principle outcome measures, significant improvements in the D-cycloserine groups as compared to placebo group were found in this repeated measure analysis. This is true for generalized avoidance of heights measures (AAVQ; F(1,19)=6.1, p≦0.02), anxiety due to heights (AAQ; F(1,19)=7.9, p≦0.01), and general attitudes towards heights (ATHI; F(1,19)=4.9, p≦0.04). These significant primary outcomes were also seen when the placebo, 50 mg and 500 mg drug doses were separated (AAVQ: F(2,18)=5.9, p≦0.01; AAQ: F(2,18)=4.0, p≦0.04; ATHI: F(2,18)=2.5, p≦0.1). These data suggest that the enhanced extinction that occurred during the initial two therapy sessions was robust and lasting, and also that it was capable of generalization to real-world height situations during the 3 months that followed the therapy.

Overall Subjective and Functional Improvement Enhanced with D-Cycloserine Augmentation

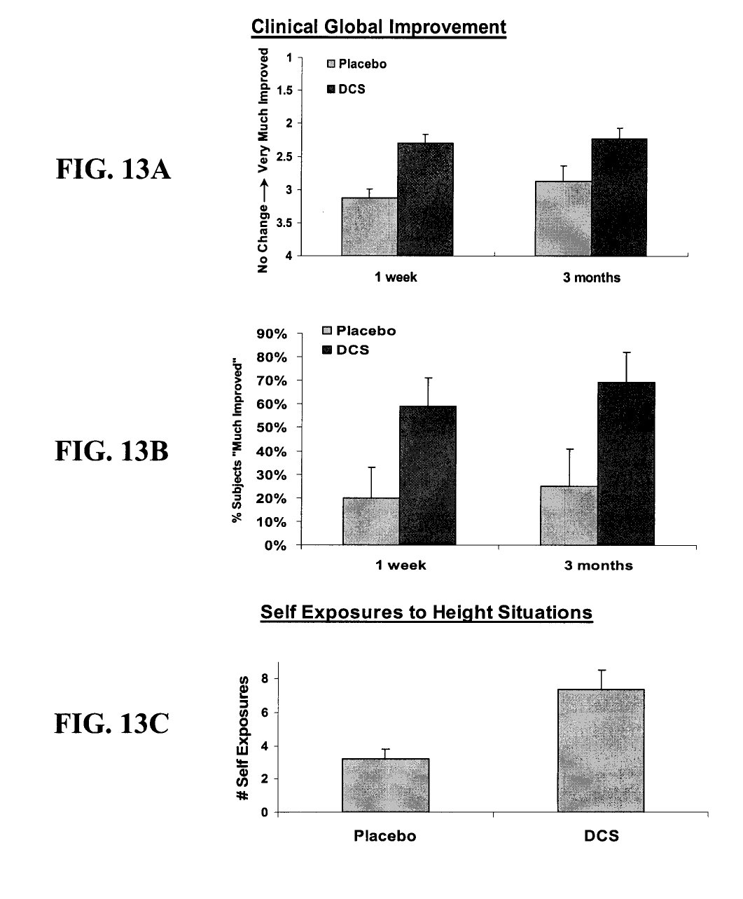

The final analyses examined general measures of overall improvement in acrophobia as well as evidence for functional gains in the subjects' lives at the 3-month follow-up assessment (FIG. 13). Average scores on the CGI scale were significantly higher at the 1 week and 3-month follow-up session as analyzed with a repeated measures ANOVA (FIG. 13A; D-cycloserine vs placebo F(1,19)=11.6, p≦0.005). Analysis of placebo, 50 mg, and 500 mg separately also revealed significant differences (F(2,18)=5.6, p≦0.01. Furthermore, as seen in FIG. 13B, the D-cycloserine group showed significantly greater percentages of subjects reporting ‘much improvement’ or ‘very much improvement’ compared to the placebo group at 1 week and 3 months (FIG. 13B; repeated measures ANOVA: overall drug effect F(1,19)=11.5, p≦0.005, but no drug x time interaction). When analyzed with drug doses separately F(2,18)=5.4, p≦0.01).

A critical measure of functional improvement is the actual number of times the subjects exposed themselves to previously fear-inducing heights in the period following the treatment. Previous studies have demonstrated that subjects successfully treated for acrophobia will expose themselves to heights in the real world following treatment much more than will those who are still fearful of heights. When subjects were asked to report the number of significant exposures (e.g. peering over a high railing, bridge, etc.) they experienced since the completion of treatment, subjects receiving D-cycloserine during treatment reported over twice as many exposures than did those receiving placebo (FIG. 13C; D-cycloserine vs placebo, F(1,18)=7.7, p≦0.01). When analyzed with drug doses separately: F(2,18)=3.6, p≦0.05.

### Discussion

These data demonstrate that D-cycloserine facilitates the effects of exposure therapy for the treatment of acrophobia. Patients in the D-cycloserine group showed some evidence of enhanced extinction after only a single dose of medication and therapy. Following two doses of medication and therapy, they showed significant reductions in levels of fear to the specific exposure environment in both subjective and objective physiological measures of fear. Finally, 3 months following the two treatment sessions, the D-cycloserine patients showed significant improvements on all general acrophobia measures, their own self-exposures in the real world, and their impression of clinical self-improvement.

The data indicate that patients receiving D-cycloserine experienced no change in anxiety or fear during the exposure paradigm so that the enhancement of extinction is not due simply to altered intensity of exposure. Additionally, the placebo and drug groups were evenly matched on all measures prior to the study (Table 2) suggesting that pre-treatment variables did not contribute to the differential improvement in groups. The slightly higher, but non-significant, depression scores in the placebo group compared to the D-cycloserine group (BDI=7.7 vs 4.2) raised the issue of whether subclinical depression could account for some of the differences seen. To test this hypothesis, all the primary outcomes were re-analyzed with pre-treatment BDI as a co-variate. In all cases (lor 3 week SUDS, SCF, AAQ, AAVQ, ATH, CGI, and self-exposure) none of the covariate analyses were significant (p's=0.12-0.88). Therefore the data presented here specifically support the role of D-cycloserine during exposure therapy contributing to the resultant enhanced improvement in acrophobia.

It is interesting to note that no apparent increase in extinction was observed during the treatment session, but only between sessions. It has been suggested that the NMDA-dependent phase of extinction training occurs during the post-extinction consolidation period (Santini E, Muller R U & Quirk G J. Consolidation of extinction learning involves transfer from NMDA-independent to NMDA-dependent memory. J Neurosci 2001; 21:9009-17). Although Specific Phobia provides an easily testable disorder that is amenable to behavioral exposure therapy, this form of therapy is also the mainstay of treatment for other anxiety disorders such as panic disorder, obsessive compulsive disorder and post-traumatic stress disorder. In addition, the process of extinction of conditioned cues is thought to be important for recovery from disorders of substance dependence (O'Brien C, Childress A, McLellan A, Ehrman R. Classical conditioning in drug-dependent humans. Ann N Y Acad Sci 1992; 654:400-15).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the representative embodiments of these concepts presented below.

1. A method for treating an individual with a psychiatric disorder, said method comprising administering to the individual in need thereof a therapeutically effective amount of a pharmacologic agent in combination with a session of psychotherapy, wherein said pharmacologic agent enhances NMDA receptor transmission in the brain, wherein said pharmacologic agent is administered to the individual only within 24 hours of psychotherapy, and wherein during said session of psychotherapy, said individual is exposed to a stimulus, and trained to develop an altered response to said stimulus.

2. The method of claim 1, wherein said psychiatric disorder is selected from the group consisting of a fear and anxiety disorder, an addictive disorder, and a mood disorder.

3. The method of claim 1, wherein the administration of said therapeutically effective amount of a pharmacologic agent occurs within about 4 hours before psychotherapy.

4. The method of claim 1, wherein said pharmacologic agent that enhances NMDA receptor transmission in the brain is a partial NMDA receptor agonist.

5. The method of claim 4, wherein said partial NMDA receptor agonist acts at the glycine modulatory site of the NMDA receptor.

6. A method for treating a fear and anxiety disorder, said method comprising administering to a patient in need thereof a therapeutically effective amount of a pharmacologic agent in combination with a session of psychotherapy, wherein said pharmacologic agent enhances NMDA receptor transmission in the brain, wherein said pharmacologic agent is administered to the patient only within 24 hours of psychotherapy, and wherein during said session of psychotherapy, the patient is exposed to a stimulus and trained to develop an altered response to said stimulus.

7. The method of claim 6, wherein said administration of said pharmacologic agent occurs within about 4 hours before psychotherapy.

8. The method of claim 6, wherein said fear and anxiety disorder is selected from the group consisting of panic disorder, specific phobia, post-traumatic stress disorder, social anxiety disorder, and obsessive-compulsive disorder.

9. An improved psychotherapy method, wherein the improvement comprises administering to a patient in need thereof a therapeutically effective amount of a pharmacologic agent that enhances extinction learning when combined with a session of psychotherapy, wherein said pharmacologic agent is administered to the patient only within 24 hours of psychotherapy, wherein said pharmacologic agent enhances NMDA receptor transmission in the brain, and wherein during said session of psychotherapy, the patient is exposed to a stimulus and trained to develop an altered response to said stimulus.
